SEQUENCE LISTING (18 sequences)

SEQ ID NO: 1 - protein, 390 aa, Homo sapiens
MPPSGLRLLPLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 2 - protein, 442 aa, Homo sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSETVCPVVTTPSGSVGSLCSRQSQVLCGYLDAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 3 - protein, 112 aa, Homo sapiens [sequence omitted]

SEQ ID NO: 4 - DNA, 336 bp, Homo sapiens
  source 1..336: mol_type = genomic DNA
gcactcgatacgaattactgttttagaaatcttgaagagaactgttgcgtgcgtcccctgtacattgatttccgtcaagatctgggatggaaatgggttcatgaaccaaagggttactatgccaacttttgttctggtccttgcccttacctgcggtcggctgatactactcattccaccgttctggggctgtacaacactctgaatcctgaagcttccgcctccccatgttgtgtccctcaagacctggagccactgaccatcctgtactacgttggacgcacccccaaagtggaacaactctccaacatggttgtcaagagctgcaaatgttca

SEQ ID NO: 5 - DNA, 939 bp, Pichia pastoris
  source 1..939: mol_type = genomic DNA
gatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 6 - DNA, 267 bp, synthetic construct
  source 1..267: mol_type = other DNA
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagct

SEQ ID NO: 7 - DNA, 247 bp, Pichia pastoris
  source 1..247: mol_type = genomic DNA
tcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgaga

SEQ ID NO: 8 - DNA, 51 bp, synthetic construct
  source 1..51: mol_type = other DNA
catcatcatcatcatcatgtgagcggctggcggctgttcaagaagattagc

SEQ ID NO: 9 - DNA, 5161 bp, synthetic construct
  source 1..5161: mol_type = other DNA
cgtgcggccgcccctgaattcgcatctagatggtagagccacaaacagccggtacaagcaacgatctccaggaccatctgaatcatgcgcggatgacacgaactcacgacggcgatcacagacattaacccacagtacagacactgcgacaacgtggcaattcgtcgcaataccgtctcactgaactggccgataattgcagacgaacggatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgtatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctggttctgcactcgatacgaattactgttttagaaatcttgaagagaactgttgcgtgcgtcccctgtacattgatttccgtcaagatctgggatggaaatgggttcatgaaccaaagggttactatgccaacttttgttctggtccttgcccttacctgcggtcggctgatactactcattccaccgttctggggctgtacaacactctgaatcctgaagcttccgcctccccatgttgtgtccctcaagacctggagccactgaccatcctgtactacgttggacgcacccccaaagtggaacaactctccaacatggttgtcaagagctgcaaatgttcaggatcccatcatcatcatcatcatgtgagcggctggcggctgttcaagaagattagctaatggctcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagagctgagcatgagacggaaatctgctcgtcagtggtgctcacactgacgaatcatgtacagatcataccgatgactgcctggcgactcacaactaagcaagacagccggaaccagcgccggcgaacaccactgcatatatggcatatcacaacagtccaactagtgcactgcagtacagtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctccagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaaaatggctaaattaacatctgccgttcctgttttaacagctagggatgttgcaggtgctgtagagttttggacagataggttaggattctcaagagactttgttgaggacgattttgctggtgttgtcagggatgacgttactttatttatctcagcagtccaagatcaagttgtccctgataatacattggcttgggtctgggtcaggggtttagatgaattatatgctgaatggtcagaagttgtatctacaaacttcagagatgcttctggtccagctatgaccgagattggtgaacagccatggggtagagaatttgctttgagagatccagctggaaattgtgttcattttgttgctgaagaacaagattaaagtaactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactggagttggccgtggccgtgctcgtcctcgtcggccggcttgtcgacgacggcggtcaccgtcgtcaggatcatccgggccacaagcttgctgacagaagcctcaagaaaaaaaaaattcttcttcgactatgctggaggcagagatgatcgagccggtagttaactatatatagctaaattggttccatcacccgagcggccgcgtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctggagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataac

SEQ ID NO: 10 - DNA, 97 bp, synthetic construct
  source 1..97: mol_type = other DNA
gcatcgtctcatcggtctcaatcccatcatcatcatcatcatgtgagcggctggcggctgttcaagaagattagctggctgagacctgagacggcat

SEQ ID NO: 11 - DNA, 100 bp, synthetic construct
  source 1..100: mol_type = other DNA
atgccgtctcaggtctcagccattagctaatcttcttgaacagccgccagccgctcacatgatgatgatgatgatgggattgagaccgatgagacgatgc

SEQ ID NO: 12 - DNA, 336 bp, Homo sapiens
  source 1..336: mol_type = genomic DNA
gctttggacaccaactactgcttcagaaacctggaagagaactgttgcgtcagaccactgtacatcgacttcagacaagaccttggttggaagtgggttcacgaacctaagggttactacgccaacttctgttctggtccatgtccatacttgagatccgctgacactactcactctaccgttttgggtctgtacaacactttgaacccagaggcttctgcttccccatgttgtgttccacaagacttggagccattgaccatcttgtactacgttggtagaaccccaaaggtcgagcagttgtccaacatggttgttaagtcctgcaagtgttct

SEQ ID NO: 13 - DNA, 336 bp, Homo sapiens
  source 1..336: mol_type = genomic DNA
gctttggatactaactactgttttagaaacttggaagaaaactgttgtgttagaccattgtacattgattttagacaagatttgggttggaagtgggttcatgaaccaaagggttactacgctaacttttgttctggtccatgtccatacttgagatctgctgatactactcattctactgttttgggtttgtacaacactttgaacccagaagcttctgcttctccatgttgtgttccacaagatttggaaccattgactattttgtactacgttggtagaactccaaaggttgaacaattgtctaacatggttgttaagtcttgtaagtgttct

SEQ ID NO: 14 - DNA, 46 bp, synthetic construct
  source 1..46: mol_type = other DNA
atccgaaaacctttattttcaaggtcatcatcaccaccatcattaa

SEQ ID NO: 15 - DNA, 46 bp, synthetic construct
  source 1..46: mol_type = other DNA
gccattaatgatggtggtgatgatgaccttgaaaataaaggttttc

SEQ ID NO: 16 - DNA, 5149 bp, synthetic construct
  source 1..5149: mol_type = other DNA
cgtgcggccgcccctgaattcgcatctagatggtagagccacaaacagccggtacaagcaacgatctccaggaccatctgaatcatgcgcggatgacacgaactcacgacggcgatcacagacattaacccacagtacagacactgcgacaacgtggcaattcgtcgcaataccgtctcactgaactggccgataattgcagacgaacggatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgtatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctggttctgctttggacaccaactactgcttcagaaacctggaagagaactgttgcgtcagaccactgtacatcgacttcagacaagaccttggttggaagtgggttcacgaacctaagggttactacgccaacttctgttctggtccatgtccatacttgagatccgctgacactactcactctaccgttttgggtctgtacaacactttgaacccagaggcttctgcttccccatgttgtgttccacaagacttggagccattgaccatcttgtactacgttggtagaaccccaaaggtcgagcagttgtccaacatggttgttaagtcctgcaagtgttctggatccgaaaacctttattttcaaggtcatcatcaccaccatcattaatggctcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagagctgagcatgagacggaaatctgctcgtcagtggtgctcacactgacgaatcatgtacagatcataccgatgactgcctggcgactcacaactaagcaagacagccggaaccagcgccggcgaacaccactgcatatatggcatatcacaacagtccaactagtgcactgcagtacagtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctccagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaaaatggctaaattaacatctgccgttcctgttttaacagctagggatgttgcaggtgctgtagagttttggacagataggttaggattctcaagagactttgttgaggacgattttgctggtgttgtcagggatgacgttactttatttatctcagcagtccaagatcaagttgtccctgataatacattggcttgggtctgggtcaggggtttagatgaattatatgctgaatggtcagaagttgtatctacaaacttcagagatgcttctggtccagctatgaccgagattggtgaacagccatggggtagagaatttgctttgagagatccagctggaaattgtgttcattttgttgctgaagaacaagattaaagtaactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactggagttggccgtggccgtgctcgtcctcgtcggccggcttgtcgacgacggcggtcaccgtcgtcaggatcatccgggccacaagcttgctgacagaagcctcaagaaaaaaaaaattcttcttcgactatgctggaggcagagatgatcgagccggtagttaactatatatagctaaattggttccatcacccgagcggccgcgtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctggagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataac

SEQ ID NO: 17 - DNA, 5149 bp, synthetic construct
  source 1..5149: mol_type = other DNA
cgtgcggccgcccctgaattcgcatctagatggtagagccacaaacagccggtacaagcaacgatctccaggaccatctgaatcatgcgcggatgacacgaactcacgacggcgatcacagacattaacccacagtacagacactgcgacaacgtggcaattcgtcgcaataccgtctcactgaactggccgataattgcagacgaacggatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgtatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctggttctgctttggatactaactactgttttagaaacttggaagaaaactgttgtgttagaccattgtacattgattttagacaagatttgggttggaagtgggttcatgaaccaaagggttactacgctaacttttgttctggtccatgtccatacttgagatctgctgatactactcattctactgttttgggtttgtacaacactttgaacccagaagcttctgcttctccatgttgtgttccacaagatttggaaccattgactattttgtactacgttggtagaactccaaaggttgaacaattgtctaacatggttgttaagtcttgtaagtgttctggatccgaaaacctttattttcaaggtcatcatcaccaccatcattaatggctcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagagctgagcatgagacggaaatctgctcgtcagtggtgctcacactgacgaatcatgtacagatcataccgatgactgcctggcgactcacaactaagcaagacagccggaaccagcgccggcgaacaccactgcatatatggcatatcacaacagtccaactagtgcactgcagtacagtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctccagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaaaatggctaaattaacatctgccgttcctgttttaacagctagggatgttgcaggtgctgtagagttttggacagataggttaggattctcaagagactttgttgaggacgattttgctggtgttgtcagggatgacgttactttatttatctcagcagtccaagatcaagttgtccctgataatacattggcttgggtctgggtcaggggtttagatgaattatatgctgaatggtcagaagttgtatctacaaacttcagagatgcttctggtccagctatgaccgagattggtgaacagccatggggtagagaatttgctttgagagatccagctggaaattgtgttcattttgttgctgaagaacaagattaaagtaactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactggagttggccgtggccgtgctcgtcctcgtcggccggcttgtcgacgacggcggtcaccgtcgtcaggatcatccgggccacaagcttgctgacagaagcctcaagaaaaaaaaaattcttcttcgactatgctggaggcagagatgatcgagccggtagttaactatatatagctaaattggttccatcacccgagcggccgcgtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctggagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataac

SEQ ID NO: 18 - DNA, 42 bp, synthetic construct
  source 1..42: mol_type = other DNA
gaaaacctttattttcaaggtcatcatcaccaccatcattaa

(19) United States
(12) Patent Application Publication
(10) Pub. No.: US 2025/0197465 A1
(43) Pub. Date: Jun. 19, 2025

(54) RECOMBINANT TRANSFORMING GROWTH FACTOR BETA IN YEAST

(71) Applicant: MULTUS BIOTECHNOLOGY LIMITED, London (GB)

(72) Inventors: George Mackford TAYLOR, London (GB); Kevin PAN, London (GB); Seong Hyun (Brandon) MA, London (GB)

(21) Appl. No.: 18/845,610

(22) PCT Filed: Mar. 10, 2023
(86) PCT No.: PCT/EP2023/056197

§ 371 Date: Sep. 10, 2024

(30) Foreign Application Priority Data
Mar. 11, 2022 (GB) 2203414.4

(51) Int. Cl. C07K 14/495; C12N 1/16; C12N 5/00; C12N 15/81; C12R 1/84

(52) U.S. Cl. CPC C07K 14/495; C12N 1/16; C12N 5/0018; C12N 15/815; C12N 2501/15; C12R 2001/84

(57) ABSTRACT

The invention relates to the expression and secretion of transforming growth factor beta (TGFβ) in methylotrophic yeast such as Pichia pastoris (P. pastoris), yeast cells whose genome comprises TGFβ, and uses thereof, including use in growth media.

### FIELD OF THE INVENTION

The invention relates to the expression and secretion of transforming growth factor beta (TGFβ) in methylotrophic yeast such as Pichia pastoris (P. pastoris), yeast cells whose genome comprises TGFβ, and uses thereof, including use in growth media.

### BACKGROUND OF THE INVENTION

Growth factors are naturally occurring cell signalling molecules that play a number of essential roles including regulating cell proliferation and development, wound healing and cellular differentiation.

Growth medium used in cell culture usually includes combinations of growth factors. It is therefore important to be able to produce growth factors suitable for use in growth medium efficiently and in a cost-effective manner. Currently, there is no such method of producing TGFβ. Methods used to produce TGFβ are labour intensive, and it is therefore an expensive material. There is a need for an efficient method of production of purified TGFβ.

In the growing field of cultivated meat, cell culture is a fundamental aspect of the process. One of the limiting steps in the production of cultivated meat is the high cost of growth media. Climate and other environmental concerns are continuing to drive the demand for cultivated meat, and therefore also the need for improved growth media to replace animal serum-based media.

Transforming growth factor beta (TGFβ) are a family of cell regulatory proteins, and members of this family are involved in a wide variety of cellular processes. TGFβ1, TGFβ2 and TGFβ3 are members of this family. TGFβ binds to specific TGFβ receptors (TGFβRs), such as TGFβR1 and TGFβR2. TGFβ is a key signalling protein that can regulate a variety of functions, including cell proliferation, differentiation, apoptosis, and metabolism. TGFβ is commonly used in growth media and there is an ongoing need for an improved method of producing TGFβ polypeptides in an efficient manner.

The present invention meets this need by providing yeast cells whose genome comprises TGFβ. To our knowledge, this is the first successful and efficient expression of TGFβ in methylotrophic yeast, such as P. pastoris.

It is particularly beneficial to express TGFβ in methylotrophic yeast, including P. pastoris, because of the cells post translational modification machinery. TGFβ3, for example, contains five disulphide bridges and will correctly fold upon formation of these disulphide bridges. TGFβ3 has three N-linked glycosylation sites, advantageously P. pastoris offers a similar glycosylation pattern to the human pattern ensuring similar folding and activity of TGFβ3, while different enough that it minimised the risk of immune responses being induced.

In addition, methylotrophic yeast, including P. pastoris, secrete recombinantly produced proteins more readily than other types of yeast, such as Saccharomyces cerevisiae, and therefore are much better suited as platforms for producing recombinant proteins.

### SUMMARY OF THE INVENTION

Provided herein is a methylotrophic yeast cell, optionally a Pichia pastoris cell, whose genome comprises a sequence encoding transforming growth factor beta.

In some embodiments, the yeast cell genome comprises a sequence encoding TGFβ1, TGFβ2 or TGFβ3.

In some embodiments, the yeast cell genome comprises a sequence encoding TGFβ3.

In some embodiments, the yeast cell has been transformed using a plasmid, wherein the plasmid comprises the nucleic acid sequence of SEQ ID NO: 7, SEQ ID NO: 14 or SEQ ID NO: 15.

In some embodiments, the yeast cell has been transformed using a plasmid, wherein the plasmid comprises the nucleic acid sequence of SEQ ID NO: 7.

In some embodiments, the yeast cell has been transformed using a plasmid, wherein the plasmid comprises the nucleic acid sequence of SEQ ID NO: 14.

In some embodiments, the yeast cell has been transformed using a plasmid, wherein the plasmid comprises the nucleic acid sequence of SEQ ID NO: 15.

Also provided herein is a polypeptide comprising the amino acid sequence of SEQ ID NO: 3, obtained or obtainable from a yeast cell of the invention.

Also provided herein is a method of producing a TGFβ polypeptide, wherein the method comprises transforming a yeast cell to express TGFβ.

Also provided herein is a method of growing an animal cell, wherein the method comprises cultivating the animal cell in a growth media containing a TGFβ polypeptide of the invention.

Also provided herein is use of a polypeptide produced according to the invention in an animal cell growth media.

### BRIEF DESCRIPTION OF THE FIGURES

FIGS. 1A and 1B show an example of a plasmid (pGT86) used to transform P. pastoris, in both circularised form (A) and linearised form (B).

FIG. 2 shows dotblot expression results.

FIG. 3 shows western blot expression analysis results.

FIGS. 4A-4D show examples of other plasmids (pBM64 and pBM65) used to transform P. pastoris, in both circularised form (A, C) and linearised form (B, D).

FIG. 5 shows dotplot expression results for plasmid pBM64.

FIG. 6 shows dotplot expression results for plasmid pBM65.

FIG. 7 shows dotplot expression results for plasmid pBM65 following liquid PVTA.

FIG. 8 shows western blot results for plasmid pBM65 following liquid PVTA.

### DETAILED DESCRIPTION OF THE INVENTION

The present invention provides novel yeast cells, whose genomes comprise TGFβ and are capable of expressing TGFβ. The present invention also provides methods of obtaining said yeast cells by transformation and polypeptides produced by these yeast cells.

Novel yeast cells of the invention include methylotrophic yeast cells such as P. pastoris cells. Methylotrophic yeasts can use methanol as a sole source of carbon and energy. All methylotrophic yeasts possess a common methanol-utilizing pathway. Methanol is first oxidized by AOX in peroxisomes to form formaldehyde and hydrogen peroxide (H₂O₂), both of which are toxic to cells and are broken down by the enzymes located in the peroxisome. Methylotrophic yeast cells achieve surprisingly high levels of protein expression in the present invention compared to non-methylotrophic yeast cells, such as Saccharomyces cells.

In an embodiment, the invention provides TGFβ coding sequences which are particularly effective in the yeast cell of the invention. In particular, such coding sequences produce surprisingly high levels of expression of TGFβ.

Unless defined otherwise, all technical and scientific terms used herein generally have the same meaning as commonly understood by a person skilled in the art to which this invention belongs. Generally, the nomenclature used herein and the laboratory procedures in cell culture, molecular genetics, organic chemistry, nucleic acid chemistry and hybridisation are those well-known and commonly employed in the art. Standard techniques are used for nucleic acid and peptide synthesis. The techniques and procedures are generally performed according to conventional methods in the art. The nomenclature used herein, and the laboratory procedures of synthetic biology described below, are those well-known and commonly employed in the art.

### Cells of the Invention

In some embodiments, the invention provides a methylotrophic yeast cell whose genome comprises transforming growth factor beta (TGFβ).

The yeast cell of the invention is a recombinant yeast cell and contains at least one nucleic acid sequence that is not naturally present in the cell.

In some embodiments, the yeast cell of the invention is a Pichia, Candida, Hansenula, Ogataea, Torulopsis or Komagataella cell.

In some embodiments, the yeast cell of the invention is a Pichia pastoris, Candida boidinii, Ogataea methanolica, Ogataea polymorpha, Ogataea thermomethanolica or Meyerozyma guilliermondii cell.

In some embodiments, the yeast cell of the invention is a Pichia pastoris cell.

Pichia pastoris is also known as Komagataella phaffii, K. phaffii, Komagataella phaffii (Pichia pastoris), Pichia pastoris (Komagataella phaffii), Komagataella kurtzmanii (K. kurtzmanii) and other such variations. These terms are intended to refer to the same species and are used interchangeably herein.

Pichia pastoris is an example of a methylotrophic yeast.

The terms ‘TGFβ1’, ‘TGFb1’, ‘TGFbeta1’, ‘TGFβ-1’, ‘TGFb-1’, ‘TGFbeta-1’, ‘transforming growth factor beta 1’ and ‘transforming growth factor beta-1’, and similar terms all refer to the same growth factor, and can be used interchangeably.

The terms ‘TGFβ2’, ‘TGFb2’, ‘TGFbeta2’, ‘TGFβ-2’, ‘TGFb-2’, ‘TGFbeta-2’, ‘transforming growth factor beta 2’ and ‘transforming growth factor beta-2’, and similar terms all refer to the same growth factor, and can be used interchangeably.

The terms ‘TGFβ3’, ‘TGFb3’, ‘TGFbeta3’, ‘TGFβ-3’, ‘TGFb-3’, ‘TGFbeta-3’, ‘transforming growth factor beta 3’ and ‘transforming growth factor beta-3’, and similar terms all refer to the same growth factor, and can be used interchangeably.

Human TGFβ1 comprises the amino acid sequence of SEQ ID NO: 1, and is shown below
MPPSGLRLLPLLLPLLWLLVLTPGRPAAGLSTCKTIDMELVKRKRIEAIRGQILSKLRLASPPSQG
EVPPGPLPEAVLALYNSTRDRVAGESAEPEPEPEADYYAKEVTRVLMVETHNEIYDKFKQSTH
SIYMFFNTSELREAVPEPVLLSRAELRLLRLKLKVEQHVELYQKYSNNSWRYLSNRLLAPSDSP
EWLSFDVTGVVRQWLSRGGEIEGFRLSAHCSCDSRDNTLQVDINGFTTGRRGDLATIHGMNR
PFLLLMATPLERAQHLQSSRHRRALDTNYCFSSTEKNCCVRQLYIDFRKDLGWKWIHEPKGYH
ANFCLGPCPYIWSLDTQYSKVLALYNQHNPGASAAPCCVPQALEPLPIVYYVGRKPKVEQLSN
MIVRSCKCS
Human TGFβ2 comprises the amino acid sequence of SEQ ID NO: 2, and is shown below
MHYCVLSAFLILHLVTVALSLSTCSTLDMDQFMRKRIEAIRGQILSKLKLTSPPEDYPEPEEVPPE
VISIYNSTRDLLQEKASRRAAACERERSDEEYYAKEVYKIDMPPFFPSETVCPVVTTPSGSVGS
LCSRQSQVLCGYLDAIPPTFYRPYFRIVRFDVSAMEKNASNLVKAEFRVFRLQNPKARVPEQRI
ELYQILKSKDLTSPTQRYIDSKVVKTRAEGEWLSFDVTDAVHEWLHHKDRNLGFKISLHCPCCT
FVPSNNYIIPNKSEELEARFAGIDGTSTYTSGDQKTIKSTRKKNSGKTPHLLLMLLPSYRLESQQ
TNRRKKRALDAAYCFRNVQDNCCLRPLYIDFKRDLGWKWIHEPKGYNANFCAGACPYLWSSD
TQHSRVLSLYNTINPEASASPCCVSQDLEPLTILYYIGKTPKIEQLSNMIVKSCKCS
Human TGFβ3 comprises the amino acid sequence of SEQ ID NO: 3, and is shown below:
ALDTNYCFRNLEENCCVRPLYIDFRQDLGWKWVHEPKGYYANFCSGPCPYLRSADTTHSTVL
GLYNTLNPEASASPCCVPQDLEPLTILYYVGRTPKVEQLSNMVVKSCKCS

TGFβ3 has a number of functions, such as cell proliferation, differentiation, wound healing, apoptosis, and metabolism.

TGFβ3 binds to transforming growth factor beta receptors (TGFβRs), stimulating the receptors. This bioactivity is required for effective function of TGFβ3 proteins.

In some embodiments, the yeast cell of the invention comprises the nucleic acid sequence of SEQ ID NO: 4, SEQ ID NO: 12 or SEQ ID NO: 13.

In some embodiments, the yeast cell of the invention comprises a sequence with at least 80% similarity to SEQ ID NO: 4, SEQ ID NO: 12 or SEQ ID NO: 13 that encodes TGFβ3. In some embodiments, the yeast cell of the invention comprises a sequence with at least 85% similarity to SEQ ID NO: 4, SEQ ID NO: 12 or SEQ ID NO: 13 that encodes TGFβ3. In some embodiments, the yeast cell of the invention comprises a sequence with at least 90% similarity to SEQ ID NO: 4, SEQ ID NO: 12 or SEQ ID NO: 13 that encodes TGFβ3. In some embodiments, the yeast cell of the invention comprises a sequence with at least 95% similarity to SEQ ID NO: 4, SEQ ID NO: 12 or SEQ ID NO: 13 that encodes TGFβ3. In some embodiments, the yeast cell of the invention comprises a sequence with at least 99% similarity to SEQ ID NO: 4, SEQ ID NO: 12 or SEQ ID NO: 13 that encodes TGFβ3.

In some embodiments, the yeast cell of the invention comprises the nucleic acid sequence of SEQ ID NO: 4.

In some embodiments, the yeast cell of the invention comprises a sequence with at least 80% similarity to SEQ ID NO: 4 that encodes TGFβ3. In some embodiments, the yeast cell of the invention comprises a sequence with at least 85% similarity to SEQ ID NO: 4 that encodes TGFβ3. In some embodiments, the yeast cell of the invention comprises a sequence with at least 90% similarity to SEQ ID NO: 4 that encodes TGFβ3. In some embodiments, the yeast cell of the invention comprises a sequence with at least 95% similarity to SEQ ID NO: 4 that encodes TGFβ3. In some embodiments, the yeast cell of the invention comprises a sequence with at least 99% similarity to SEQ ID NO: 4 that encodes TGFβ3.

In some embodiments, the yeast cell of the invention comprises the nucleic acid sequence of SEQ ID NO: 12.

In some embodiments, the yeast cell of the invention comprises a sequence with at least 80% similarity to SEQ ID NO: 12 that encodes TGFβ3. In some embodiments, the yeast cell of the invention comprises a sequence with at least 85% similarity to SEQ ID NO: 12 that encodes TGFβ3. In some embodiments, the yeast cell of the invention comprises a sequence with at least 90% similarity to SEQ ID NO: 12 that encodes TGFβ3. In some embodiments, the yeast cell of the invention comprises a sequence with at least 95% similarity to SEQ ID NO: 12 that encodes TGFβ3. In some embodiments, the yeast cell of the invention comprises a sequence with at least 99% similarity to SEQ ID NO: 12 that encodes TGFβ3.

In some embodiments, the yeast cell of the invention comprises the nucleic acid sequence of SEQ ID NO: 13.

In some embodiments, the yeast cell of the invention comprises a sequence with at least 80% similarity to SEQ ID NO: 13 that encodes TGFβ3. In some embodiments, the yeast cell of the invention comprises a sequence with at least 85% similarity to SEQ ID NO: 13 that encodes TGFβ3. In some embodiments, the yeast cell of the invention comprises a sequence with at least 90% similarity to SEQ ID NO: 13 that encodes TGFβ3. In some embodiments, the yeast cell of the invention comprises a sequence with at least 95% similarity to SEQ ID NO: 13 that encodes TGFβ3. In some embodiments, the yeast cell of the invention comprises a sequence with at least 99% similarity to SEQ ID NO: 13 that encodes TGFβ3.

Percent similarity (or ‘percentage similarity’) between two sequences can be calculated by multiplying the number of matches in the pair by 100 and dividing by the length of the aligned region, including gaps. Identity scoring only counts perfect matches. Gaps at the end of sequences are not included, and internal gaps are included in the length. BLAST (NCBI) can be used to produce an alignment.

In some embodiments, SEQ ID NO: 4 encodes TGFβ3 that binds at least one transforming growth factor beta receptor (TGFβR).

In some embodiments, SEQ ID NO: 4 encodes TGFβ3 that binds TGFβR1, TGFβR2 and/or TGFβR3.

In some embodiments, SEQ ID NO: 12 encodes TGFβ3 that binds at least one transforming growth factor beta receptor (TGFβR).

In some embodiments, SEQ ID NO: 12 encodes TGFβ3 that binds TGFβR1, TGFβR2 and/or TGFβR3.

In some embodiments, SEQ ID NO: 13 encodes TGFβ3 that binds at least one transforming growth factor beta receptor (TGFβR).

In some embodiments, SEQ ID NO: 13 encodes TGFβ3 that binds TGFβR1, TGFβR2 and/or TGFβR3.

In some embodiments, the yeast cell of the invention expresses a polypeptide comprising the amino acid sequence of SEQ ID NO: 1, or a fragment, analog or derivative thereof that binds at least one transforming growth factor beta receptor (TGFβR).

In some embodiments, the yeast cell of the invention expresses a polypeptide comprising the amino acid sequence of SEQ ID NO: 2, or a fragment, analog or derivative thereof that binds at least one transforming growth factor beta receptor (TGFβR).

In some embodiments, the yeast cell of the invention expresses a polypeptide comprising the amino acid sequence of SEQ ID NO: 3, or a fragment, analog or derivative thereof that binds at least one transforming growth factor beta receptor (TGFβR).

In some embodiments, the yeast cell of the invention expresses a polypeptide comprising a sequence at least 80% similarity to SEQ ID NO: 1, wherein the polypeptide binds at least one transforming growth factor beta receptor (TGFβR). In some embodiments, the polypeptide of the invention comprises a sequence with at least 85% similarity to SEQ ID NO: 1. In some embodiments, the polypeptide of the invention comprises a sequence with at least 90% similarity to SEQ ID NO: 1, wherein the polypeptide binds at least one transforming growth factor beta receptor (TGFβR). In some embodiments, the polypeptide of the invention comprises a sequence with at least 95% similarity to SEQ ID NO: 1, wherein the polypeptide binds at least one transforming growth factor beta receptor (TGFβR).

In some embodiments, the yeast cell of the invention expresses a polypeptide comprising a sequence at least 80% similarity to SEQ ID NO: 2, wherein the polypeptide binds at least one transforming growth factor beta receptor (TGFβR). In some embodiments, the polypeptide of the invention comprises a sequence with at least 85% similarity to SEQ ID NO: 2. In some embodiments, the polypeptide of the invention comprises a sequence with at least 90% similarity to SEQ ID NO: 2, wherein the polypeptide binds at least one transforming growth factor beta receptor (TGFβR). In some embodiments, the polypeptide of the invention comprises a sequence with at least 95% similarity to SEQ ID NO: 2, wherein the polypeptide binds at least one transforming growth factor beta receptor (TGFβR).

In some embodiments, the yeast cell of the invention expresses a polypeptide comprising a sequence at least 80% similarity to SEQ ID NO: 3, wherein the polypeptide binds at least one transforming growth factor beta receptor (TGFβR). In some embodiments, the polypeptide of the invention comprises a sequence with at least 85% similarity to SEQ ID NO: 3. In some embodiments, the polypeptide of the invention comprises a sequence with at least 90% similarity to SEQ ID NO: 3, wherein the polypeptide binds at least one transforming growth factor beta receptor (TGFβR). In some embodiments, the polypeptide of the invention comprises a sequence with at least 95% similarity to SEQ ID NO: 3, wherein the polypeptide binds at least one transforming growth factor beta receptor (TGFβR).

In some embodiments, the yeast cell of the invention expresses a polypeptide that binds TGFβR1, TGFβR2 and/or TGFβR3.

In some embodiments, the TGFβ3 comprised by a yeast cell of the invention is human TGFβ3.

In some embodiments, the TGFβ3 is bovine TGFβ3. In some embodiments, the TGFβ3 is porcine TGFβ3. In some embodiments, the TGFβ3 is avian TGFβ3.

In some embodiments, the TGFβ3 comprises the nucleic acid sequence of SEQ ID NO: 4, SEQ ID NO: 12 or SEQ ID NO: 13.

In some embodiments, the TGFβ3 comprises the nucleic acid sequence of SEQ ID NO: 4.

In some embodiments, the TGFβ3 comprises the nucleic acid sequence of SEQ ID NO: 12.

In some embodiments, the TGFβ3 comprises the nucleic acid sequence of SEQ ID NO: 13.

In some embodiments, the sequence encoding TGFβ3 comprises the nucleic acid sequence of SEQ ID NO: 4, SEQ ID NO: 12 or SEQ ID NO: 13.

In some embodiments, the sequence encoding TGFβ3 comprises the nucleic acid sequence of SEQ ID NO: 4.

In some embodiments, the sequence encoding TGFβ3 comprises the nucleic acid sequence of SEQ ID NO: 12.

In some embodiments, the sequence encoding TGFβ3 comprises the nucleic acid sequence of SEQ ID NO: 13.

In some embodiments, the yeast cell has been transformed with a plasmid comprising sequence encoding TGFβ1, TGFβ2 or TGFβ3.

In some embodiments, the plasmid comprises a promoter.

In some embodiments, the plasmid comprises a coding sequence.

In some embodiments, the plasmid comprises a terminator.

In some embodiments, the plasmid comprises a promoter, a coding sequence and a terminator.

In some embodiments, the plasmid comprises a signal peptide.

In some embodiments, the plasmid comprises a promoter, a signal peptide, a coding sequence and a terminator.

In some embodiments, the promoter is an alcohol oxidase I (AOX1) promoter. In some embodiments, the promoter comprises the nucleic acid sequence of SEQ ID NO: 5.

In some embodiments, the signal peptide is an alpha mating-factor signal peptide. In some embodiments, the signal peptide comprises the nucleic acid sequence of SEQ ID NO: 6.

In some embodiments, the coding sequence comprises TGFβ3, optionally human TGFβ3.

In some embodiments, the coding sequence comprises the nucleic acid sequence of SEQ ID NO: 4, SEQ ID NO: 12 or SEQ ID NO: 13.

In some embodiments, the coding sequence comprises TGFβ3, optionally human TGFβ3. In some embodiments, the coding sequence comprises the nucleic acid sequence of SEQ ID NO: 4.

In some embodiments, the coding sequence comprises TGFβ3, optionally human TGFβ3. In some embodiments, the coding sequence comprises the nucleic acid sequence of SEQ ID NO: 12.

In some embodiments, the coding sequence comprises TGFβ3, optionally human TGFβ3. In some embodiments, the coding sequence comprises the nucleic acid sequence of SEQ ID NO: 13.

In some embodiments, the terminator is an alcohol oxidase I (AOX1) terminator. In some embodiments, the terminator comprises the nucleic acid sequence of SEQ ID NO: 7,

In some embodiments, the plasmid comprises the nucleic acid sequences of SEQ ID Nos: 4-7.

In some embodiments, the plasmid comprises the nucleic acid sequences of SEQ ID NOs: 12 and 5-7.

In some embodiments, the plasmid comprises the nucleic acid sequences of SEQ ID NOs: 13 and 5-7.

In some embodiments, the plasmid comprises at least one tag.

In some embodiments, the tag comprises a His-tag fused to a HiBiT tag. In some embodiments, the tag comprises the nucleic acid sequence of SEQ ID NO: 8.

In some embodiments, the tag comprises a His-tag fused to a TEV cleavage site. In some embodiments, the tag comprises the nucleic acid sequence of SEQ ID NO: 18.

In some embodiments, the plasmid comprises the nucleic acid sequences of SEQ ID NOs: 4-8.

In some embodiments, the plasmid comprises the nucleic acid sequences of SEQ ID NOs: 12, 5-7 and 18.

In some embodiments, the plasmid comprises the nucleic acid sequences of SEQ ID NOs: 13, 5-7 and 18.

In some embodiments, the plasmid comprises the nucleic acid sequence of SEQ ID NO: 9, SEQ ID NO: 16 or SEQ ID NO: 17.

In some embodiments, the plasmid comprises the nucleic acid sequence of SEQ ID NO: 9.

In some embodiments, the plasmid comprises the nucleic acid sequence of SEQ ID NO: 16.

In some embodiments, the plasmid comprises the nucleic acid sequence of SEQ ID NO: 17.

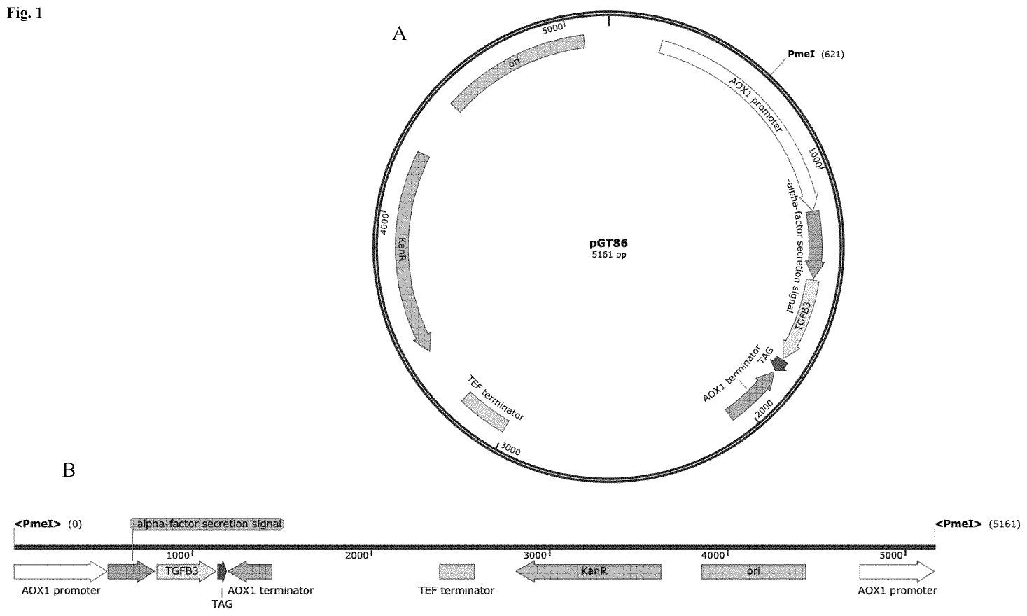

In some embodiments, the plasmid is the plasmid shown in FIG. 1A.

In some embodiments, the plasmid is the plasmid shown in FIG. 1B.

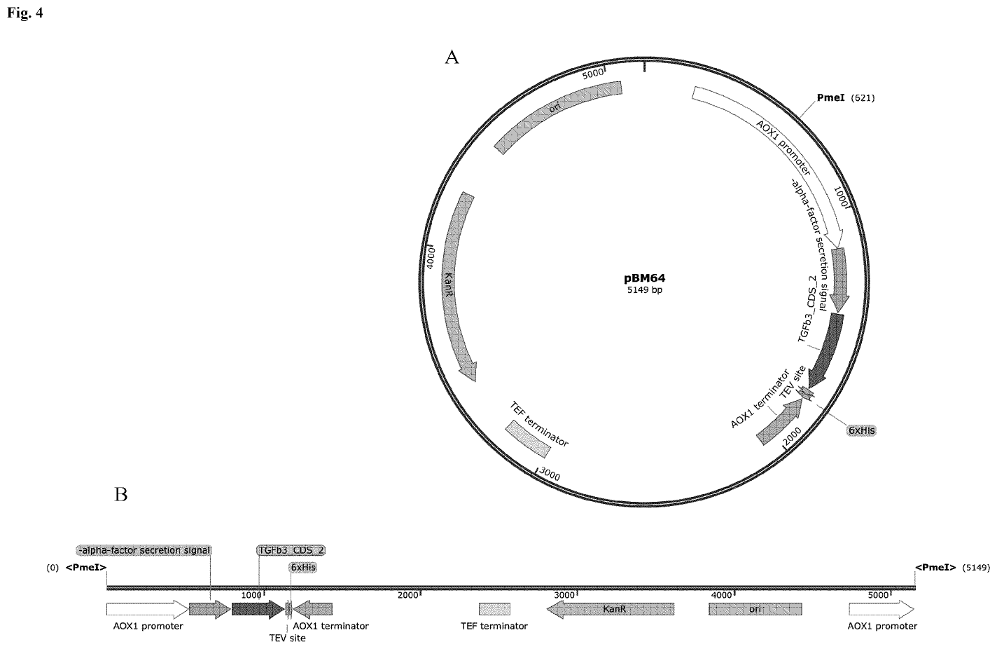

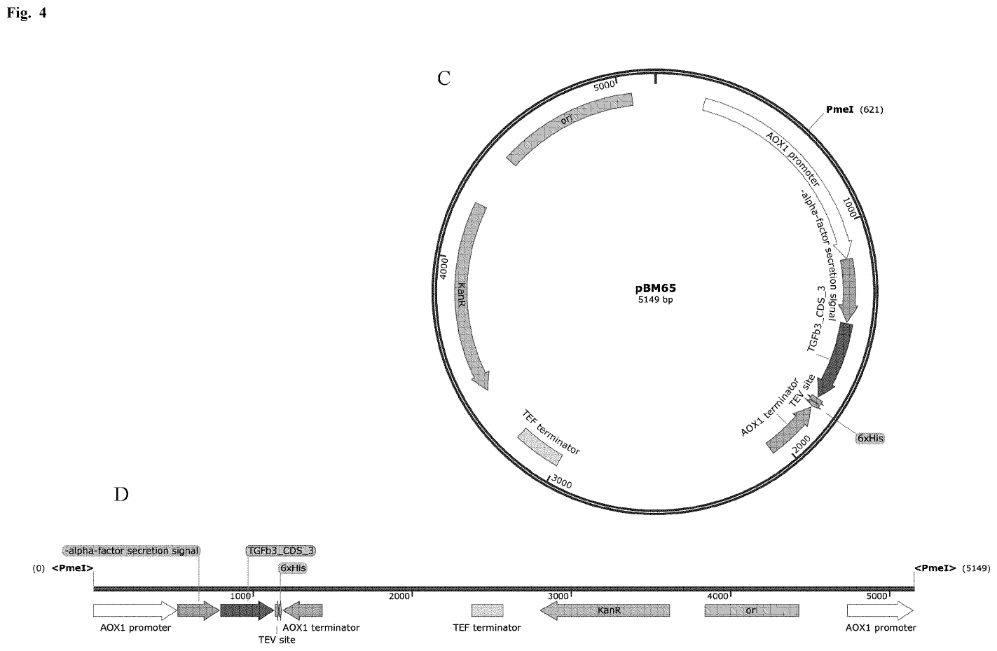

In some embodiments, the plasmid is the plasmid shown in FIG. 4A.

In some embodiments, the plasmid is the plasmid shown in FIG. 4B.

In some embodiments, the plasmid is the plasmid shown in FIG. 4C.

In some embodiments, the plasmid is the plasmid shown in FIG. 4D.

The plasmid described herein may be circular.

The plasmid described herein may be linearised.

Polypeptides Obtained or Obtainable from a Yeast Cell of the Invention

In some embodiments, the invention provides a polypeptide obtained or obtainable from a yeast cell disclosed herein.

In some embodiments, the polypeptide of the invention comprises the amino acid sequence of SEQ ID NO: 1, SEQ ID NO: 2 or SEQ ID NO: 3.

In some embodiments, the polypeptide of the invention comprises a sequence at least 80% similarity to SEQ ID NO: 1, wherein the polypeptide binds at least one transforming growth factor beta receptor (TGFβR). In some embodiments, the polypeptide of the invention comprises a sequence with at least 85% similarity to SEQ ID NO: 1, wherein the polypeptide binds at least one transforming growth factor beta receptor (TGFβR). In some embodiments, the polypeptide of the invention comprises a sequence with at least 90% similarity to SEQ ID NO: 1, wherein the polypeptide binds at least one transforming growth factor beta receptor (TGFβR). In some embodiments, the polypeptide of the invention comprises a sequence with at least 95% similarity to SEQ ID NO: 1, wherein the polypeptide binds at least one transforming growth factor beta receptor (TGFβR).

In some embodiments, the polypeptide of the invention comprises a sequence at least 80% similarity to SEQ ID NO: 2, wherein the polypeptide binds at least one transforming growth factor beta receptor (TGFβR). In some embodiments, the polypeptide of the invention comprises a sequence with at least 85% similarity to SEQ ID NO: 2, wherein the polypeptide binds at least one transforming growth factor beta receptor (TGFβR). In some embodiments, the polypeptide of the invention comprises a sequence with at least 90% similarity to SEQ ID NO: 2, wherein the polypeptide binds at least one transforming growth factor beta receptor (TGFβR). In some embodiments, the polypeptide of the invention comprises a sequence with at least 95% similarity to SEQ ID NO: 2, wherein the polypeptide binds at least one transforming growth factor beta receptor (TGFβR).

In some embodiments, the polypeptide of the invention comprises a sequence at least 80% similarity to SEQ ID NO: 3, wherein the polypeptide binds at least one transforming growth factor beta receptor (TGFβR). In some embodiments, the polypeptide of the invention comprises a sequence with at least 85% similarity to SEQ ID NO: 3, wherein the polypeptide binds at least one transforming growth factor beta receptor (TGFβR). In some embodiments, the polypeptide of the invention comprises a sequence with at least 90% similarity to SEQ ID NO: 3, wherein the polypeptide binds at least one transforming growth factor beta receptor (TGFβR). In some embodiments, the polypeptide of the invention comprises a sequence with at least 95% similarity to SEQ ID NO: 3, wherein the polypeptide binds at least one transforming growth factor beta receptor (TGFβR).

Percent similarity (or ‘percentage similarity’) between two sequences can be calculated by multiplying the number of matches in the pair by 100 and dividing by the length of the aligned region, including gaps. Identity scoring only counts perfect matches and does not consider the degree of similarity of amino acids to one another. Gaps at the end of sequences are not included, and internal gaps are included in the length.

The present invention further relates to fragments, analogs and derivatives of a polypeptide obtained or obtainable from a yeast cell disclosed therein, where the “fragment,” “derivative” and “analog” retains essentially the same biological function or activity as a polypeptide as set forth in SEQ ID NO: 1, SEQ ID NO: 2 or SEQ ID NO: 3.

### Methods of the Invention

In some embodiments, a method of producing a TGFβ polypeptide is provided, wherein the method comprises cultivating the yeast cell of the invention under conditions which allow for expression of said polypeptide and, optionally, recovering the expressed polypeptide.

In some embodiments, the method of producing of a TGFβ polypeptide further comprises i) optimising a nucleic acid encoding a TGFβ polypeptide for expression in yeast, ii) screening more than ten clones for expression of TGFβ and/or iii) detecting expression with a tag.

In some embodiments, the method of producing of a TGFβ polypeptide further comprises i) optimising a nucleic acid encoding a TGFβ polypeptide for expression in yeast, ii) screening more than ten clones for expression of TGFβ and/or iii) detecting expression with a HiBiT peptide tag.

In some embodiments, the method of producing of a TGFβ polypeptide further comprises i) optimising a nucleic acid encoding a TGFβ polypeptide for expression in yeast, ii) screening more than ten clones for expression of TGFβ and/or iii) detecting expression with a His-tag.

In some embodiments, the invention provides a method of producing a TGFβ3 polypeptide comprising transforming a methylotrophic yeast with a linearised plasmid comprising the nucleic acid sequences of SEQ ID NOs: 4-7 by electroporation and screening more than 10 clones for expression.

In some embodiments, the invention provides a method of producing a TGFβ3 polypeptide comprising transforming a methylotrophic yeast with a linearised plasmid comprising the nucleic acid sequences of SEQ ID NOs: 12 and 5-7 by electroporation and screening more than 10 clones for expression.

In some embodiments, the invention provides a method of producing a TGFβ3 polypeptide comprising transforming a methylotrophic yeast with a linearised plasmid comprising the nucleic acid sequences of SEQ ID NOs: 13 and 5-7 by electroporation and screening more than 10 clones for expression.

In some embodiments, the invention provides a method of producing a TGFβ3 polypeptide comprising transforming Pichia pastoris with a linearised plasmid comprising the nucleic acid sequences of SEQ ID NOs: 4-7 by electroporation and screening more than 10 clones for expression.

In some embodiments, the invention provides a method of producing a TGFβ3 polypeptide comprising transforming Pichia pastoris with a linearised plasmid comprising the nucleic acid sequences of SEQ ID NOs: 12 and 5-7 by electroporation and screening more than 10 clones for expression.

In some embodiments, the invention provides a method of producing a TGFβ3 polypeptide comprising transforming Pichia pastoris with a linearised plasmid comprising the nucleic acid sequences of SEQ ID NOs: 13 and 5-7 by electroporation and screening more than 10 clones for expression.

In some embodiments, over 20 clones or over 30 clones are screened for expression. In a preferred embodiment, over 40 clones are screened.

In some embodiments, the invention comprises screening clones for expression by detection of a tag.

In some embodiments, the invention comprises screening clones for expression by detection of a HiBiT tag.

In some embodiments, the invention comprises screening clones for expression by detection of a His-tag.

In some embodiments, the method of the invention comprises growing yeast cells of the invention in baffled shake flasks.

In some embodiments, the method of the invention comprises culturing yeast cells of the invention in a stirred bioreactor.

In some embodiments, the yeast cell of the invention is cultured in fermentation media in a stirred bioreactor.

In some embodiments, the yeast cell of the invention is cultured in fermentation media in a stirred bioreactor for a defined time, a defined pH, and/or a defined temperature.

In some embodiments, the defined time is between 6-144 hours, preferably between 24-96 hours.

In some embodiments, the defined pH is between 2.0-10.0, preferably between 4.0-8.0.

In some embodiments, the defined temperature is between 4-37° C., preferably between 8-30° C.

In some embodiments, TGFβ is expressed and secreted out of the yeast cell and into the fermentation media. In some embodiments, the TGFβ is not stored intracellularly in the yeast cell. Thus, the present invention also relates to secreted expression of TGFβ using yeast cells.

In some embodiments, following the cultivation period, the TGFβ-containing supernatant is separated from the yeast cells by centrifugation.

In some embodiments, following the cultivation period, the TGFβ-containing supernatant is separated from the yeast cells by centrifugation and subjected to filtration-based purification.

In some embodiments, the filtration-based purification removes unwanted molecules.

In some embodiments, the filtered TGFβ-containing sample will be freeze-dried, optionally for long term stable storage.

It will be understood that the invention can relate to expression of functional variants of TGFβ1, TGFβ2 and TGFβ3, and proteins in the same biological pathway, which achieve the same, or similar, downstream effect to wild-type TGFβ.

For example, the invention can also relate to expression of any activator of a TGFβ family receptor. The invention also can also relate expression of an activator of the TGFβ3 receptor. The invention also relates to the formation of TGF-beta-3-TGFR complex (EMBL-EBI ComplexAc: CPX-2544). The invention also relates to an activator of the TGFβ signalling pathway. The invention also relates to an activator of the Transforming growth factor beta receptor signalling pathway (EMBL-EBI GO:0007179).

### Uses of Polypeptides Produced According to the Invention

In some embodiments, the invention provides use of the polypeptide described herein in a growth media. In some embodiments, the growth media is an animal growth media. The growth media may be serum-free.

A growth media is a medium used for the viability, growth and/or storage of cells. In some embodiments, the growth media of the invention is used for culture of fibroblasts, myoblasts, adipocytes, mesenchymal stem cells or induced pluripotent stem cells (iPSCs).

A growth media of the invention can additionally comprise one or more additional growth factors, serum or serum replacement, one or more hormones, one or more antibiotics, one or more trace elements and/or one or more antioxidants.

In some embodiments, the invention provides a method of growing a cell, wherein the method comprises cultivating the cell in a growth media containing the polypeptide described herein. In some embodiments, the cell is an animal cell. In some embodiments the growth media is an animal growth media.

It will be understood that particular embodiments described herein are shown by way of illustration and not as limitations of the invention. The principal features of this invention can be employed in various embodiments without departing from the scope of the invention. Those skilled in the art will recognise, or be able to ascertain using no more than routine study, numerous equivalents to the specific procedures described herein. Such equivalents are considered to be within the scope of this invention and are covered by the claims. All publications mentioned in the specification are indicative of the level of skill of those skilled in the art to which this invention pertains.

All publications are herein incorporated by reference to the same extent as if each individual publication was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the inherent variation of error for the feature in the below.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps

The term “or combinations thereof” as used herein refers to all permutations and combinations of the listed items preceding the term. For example, “A, B, C, or combinations thereof is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

Any part of this disclosure may be read in combination with any other part of the disclosure, unless otherwise apparent from the context.

All of the cells, polypeptides, nucleic acids and methods disclosed and claimed herein can be made and executed without undue experimentation in light of the present disclosure. While the invention has been described in terms of preferred embodiments, it will be apparent to those of skill in the art that variations may be applied to the cells, polypeptides, nucleic acids and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

The invention can be further described in the following numbered clauses:
1. A yeast cell whose genome comprises a sequence encoding transforming growth factor beta 3 (TGFβ3).
2. The yeast cell of claim 1, wherein the TGFβ3 is human TGFβ3.
3. The yeast cell of claim 1 or claim 2, wherein the TGFβ3 comprises the nucleic acid sequence of SEQ ID NO: 4.
4. The yeast cell of any preceding claim, wherein the yeast cell is a Pichia pastoris cell.
5. The yeast cell of any preceding claim, wherein the yeast cell has been transformed using a plasmid.
6. The yeast cell of claim 5, wherein the plasmid comprises a promoter, the coding sequence, and a terminator.
7. The yeast cell of claim 6, wherein the plasmid additionally comprises a signal peptide.
8. The yeast cell of claim 6 or claim 7, wherein the promoter is an alcohol oxidase I (AOX1) promoter.
9. The yeast cell of any of claim 6-8, wherein the promoter comprises the nucleic acid sequence of SEQ ID NO: 5.
10. The yeast cell of any of claims 7-9, wherein the signal peptide is an alpha mating-factor signal peptide.
11. The yeast cell of any of claims 7-10, wherein the signal peptide comprises the nucleic acid sequence of SEQ ID NO: 6.
12. The yeast cell of any of claims 6-11, wherein the terminator is an alcohol oxidase I (AOX1) terminator.
13. The yeast cell of any of claims 6-12, wherein the terminator comprises the nucleic acid sequence of SEQ ID NO: 7.
14. The yeast cell of any of claims 6-13, wherein the plasmid comprises the nucleic acid sequences of SEQ ID NOs: 4-7.
15. The yeast cell of any of claims 6-14, wherein the plasmid comprises the nucleic acid sequence of SEQ ID NO: 9.
16. A yeast cell capable of expressing TGFβ3.
17. The yeast cell of claim 16, wherein the yeast cell is a Pichia pastoris cell.
18. A polypeptide comprising the amino acid sequence of SEQ ID NO: 3, obtained or obtainable from the yeast cell of any preceding claim.
19. A method of producing a TGFβ3 polypeptide, wherein the method comprises transforming a yeast cell with a plasmid encoding TGFβ3.
20. A method of producing a TGFβ3 polypeptide, wherein the method comprises cultivating the yeast cell of any of claims 1-17 under conditions which allow for expression of said polypeptide and, optionally, recovering the expressed polypeptide.
21. The method of claim 20, further comprising i) optimising a nucleic acid encoding a TGFβ3 polypeptide for expression in yeast, optionally Pichia pastoris, ii) screening more than ten clones for expression of TGFβ3, optionally at least twenty clones, and/or iii) detecting expression with a HiBiT peptide tag.
22. A method of growing an animal cell, wherein the method comprises cultivating the animal cell in a growth media containing the polypeptide of claim 18.
23. Use of the polypeptide of claim 18 in an animal cell growth media.

The present invention is described in more detail in the following non limiting exemplification.

### EXAMPLES

The following examples will be useful in demonstrating the present invention.

### Example 1: Plasmid Assembly

Escherichia coli strain DH5a was used for all DNA assembly and all other cloning described. E. coli DH5α cells were routinely grown in LB medium (tryptone 10 g l⁻¹, yeast extract 5 g l⁻¹ and NaCl 5 g l⁻¹) at 37° C., with shaking at 225 rpm, or on LB agar plates (containing 15 g l⁻¹ bacteriological agar) at 37° C. LB was supplemented with kanamycin (50 μg ml⁻¹) or chloramphenicol (25 μg ml⁻¹) as appropriate. Chemically competent E. coli DH5α cells were routinely used for transformation.

Previously assembled plasmids used in this study are as described in Obst el al. “A Modular Toolkit for Generating Pichia pastoris Secretion Libraries”, ACS Synth. Biol., 2017 and in Lee et al. “A Highly Characterized Yeast Toolkit for Modular, Multipart Assembly”, ACS Synth. Biol., 2015.

Plasmid pGT86 was assembled from five individual parts: Paox1 (pPTK001), α-MF (pPTK005), TGFβ3 CDS (pGT2), 6xHis-tag-HiBiT (pMMp014) and Taox1(pMMp010), into the backbone pMMc002. The Golden Gate reaction was performed as described in Taylor et al. “Start-Stop Assembly: a functionally scarless DNA assembly system optimized for metabolic engineering”, NucleicAcids Res., 2019. Chemically competent E. coli cells were transformed with 10 μl of the pGT86 Golden Gate assembly reaction. The plasmids of transformant colonies were purified and sequence verified.

The TGFβ3 CDS (pGT2), 6xHis-tag-HiBiT (pMMp014) and Taox1(pMMp010) were stored in the Level 0 storage plasmid pYTK001.

The coding sequence (SEQ ID NO: 4) of the human Transforming growth factor beta isoform 3 (TGFβ3) was codon optimised for Pichia pastoris using TWIST Biosciences software (rather than optimised for the host from which it was derived, as would be normal practice) and synthesized by TWIST Biosciences. The synthetic DNA was directly mixed with pYTK001 in a Golden Gate reaction using the conditions described by Lee et al. “A Highly Characterized Yeast Toolkit for Modular, Multipart Assembly” ACS Sythn Biol., 2015. E. coli was transformed with the resultant reaction mixture. The plasmid (pGT2) of transformant colonies were purified and sequence verified.

The tags were generated by annealing complementary pairs of oligonucleotides, oBM37 and oBM38 (sequences shown below), as described in Taylor et al. The resultant double stranded DNA was mixed with pYTK001 in a Golden Gate reaction using the conditions described by Lee et al. E. coli was transformed with the resultant reaction mixture. The plasmid (pMMp014) of transformant colonies were purified and sequence verified.

The DNA sequence for the terminator Taox1 was synthesized using TWIST Biosciences. The synthetic DNA was directly mixed with pYTK001 in a Golden Gate reaction using the conditions described by Lee et al. E. coli was transformed with the resultant reaction mixture. The plasmid (pMMp010) of transformant colonies were purified and sequence verified.

oBM37
(SEQ ID NO: 10)
GCATCGTCTCATCGGTCTCAATCCCATCATCATCATCATCATGTGAGCG
GCTGGCGGCTGTTCAAGAAGATTAGCTGGCTGAGACCTGAGACGGCAT
oBM38
(SEQ ID NO: 11)
ATGCCGTCTCAGGTCTCAGCCATTAGCTAATCTTCTTGAACAGCCGCCA
GCCGCTCACATGATGATGATGATGATGGGATTGAGACCGATGAGACGAT
GC

pMMc002 was assembled using pYTK002, pYTK047, pYTK072, pYTK080 and pYTK084 via Golden Gate assembly using the reaction conditions described by Lee et al. E. coli was transformed with the resultant reaction mixture. The plasmid (pMMc002) of transformant colonies were purified and sequence verified.

The resultant plasmid is shown in FIGS. 1A and 1B.

### Example 2: Pichia pastoris Transformation

Pichia pastoris strain CBS7435 was used for transforming growth factor beta isoform 3 (TGFβ3) expression. P. pastoris cells were routinely grown in YPD medium (1% yeast extract, 2% peptone, 2% dextrose) at 30° C., with shaking at 225 rpm, or on YPD agar plates (containing 15 g l⁻¹ bacteriological agar) at 30° C. YPD was supplemented with Zeocin (100 μg ml⁻¹) as appropriate.

1000 μg of pGT86 was linearised using Pmel (NEB), as per manufacturers instructions. Linearised pGT86 was cleaned-up by gel purification using the QlAquick Gel Extraction kit (Qiagen). P. pastoris cells were transformed with 100 μg of linearised pGT86 using the condensed protocol for electroporation as described in Lin-Cereghino, J et al. “Condensed protocol for competent cell preparation and transformation of the methylotrophic yeast Pichia pastoris” Biotechniques, 2005.

### Example 3: TGFβ3 Expression

P. pastoris protein expression was carried out in buffered dextrose/methanol complex medium (BMDY/BMMY; 1% yeast extract, 2% peptone, 100 mM potassium phosphate (pH 6.0), 1.34% yeast nitrogen base, 4×10⁻⁵% biotin, 2% dextrose or 0.5% methanol).

For high throughput screening of protein expression in P. pastoris deep 96-well plates sealed with sterile breathable sealing film (Breathe Easier, Sigma Aldrich) containing 300 μl of medium were used. For larger scale expression, 500 ml baffled shake flasks containing 100 ml of medium were used. Isolated transformant clones of P. pastoris were used to inoculate YPD supplemented with zeocin and grown at 30° C. at 225 rpm for 24 hours. Cultures were diluted 1:100 into BMDY supplemented with zeocin and grown at 30° C. at 225 rpm for 24 hours. Following incubation, cells were pelleted at 4000 rpm for 5 minutes, supernatant was removed, and fresh BMMY media added to induce protein expression. Cultures were incubated at 30° C., 225 rpm for 48 hours, at 24 hours cultures were supplemented with methanol (100%) to a final concentration 0.5% v/v. Following incubation, cultures were pelleted at 4000 rpm for 5 min, and the supernatants were collected for protein expression assays.

Trichloroacetic acid (100%) was added to supernatant to a final concentration of 10%. Supernatant was incubated at −20° C. for 15 minutes, before protein precipitates were pelleted at 10,000 g, 4° C. for 15 minutes. The pellet was washed twice with acetone (100%) and then air dried. The washed pellet was resuspended in 1/10 of the supernatant volume in Tris-HCl (50 mM, pH 8).

100 μl of supernatant was loaded into individual wells of the Dot Blotter apparatus (Wolflabs). A vacuum was applied to transfer the proteins onto a nitrocellulose membrane (Amersham). TGFβ3 was detected using a 1:1000 dilution of the Mouse anti-HiBiT primary antibody (CS2006A01) followed by 1:2500 dilution of the horseradish peroxidase (HRP) conjugated anti-mouse secondary antibody (W4021). Dots were observed using the ECL Western Blotting Substrate (Promega W1001).

20 μl of sample (supernatant or concentrated supernatant) were denatured by boiling for 10 minutes in reducing SDS sample buffer (4× Laemmli protein sample buffer supplemented with 50 mM DTT). The prestained protein ladder (10-250 kDa, PageRuler, Thermo Scientific) was used to estimate molecular weight. Proteins were resolved by sodium dodecyl sulphate polyacrylamide gel electrophoresis (SDS-PAGE) using 12% Surepage precast gels (GenScript). Proteins were fixed to a nitrocellulose membrane (Amersham) using the wet/tank blotting systems (Bio Rad; 100V, 1 hour, 4° C.). TGFβ3 was detected using a 1:1000 dilution of the Mouse anti-HiBiT primary antibody (Promega CS2006A01) followed by 1:2500 dilution of the horseradish peroxidase (HRP) conjugated anti-mouse secondary antibody (Promega W4021). Dots were observed using the ECL Western Blotting Substrate (Promega W1001).

In this experimental protocol, contrary to usual practice (6-10 verified clones as recommended by the Invitrogen Pichia Expression Kit manual), we screened greater than 50 transformant clones. This was significant because only 6.25% of clones screened expressed TGFβ3.

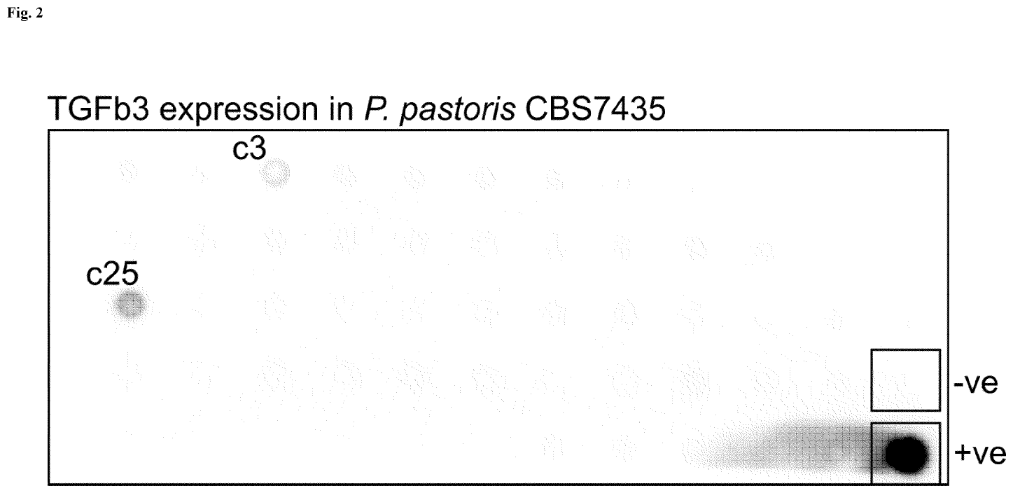

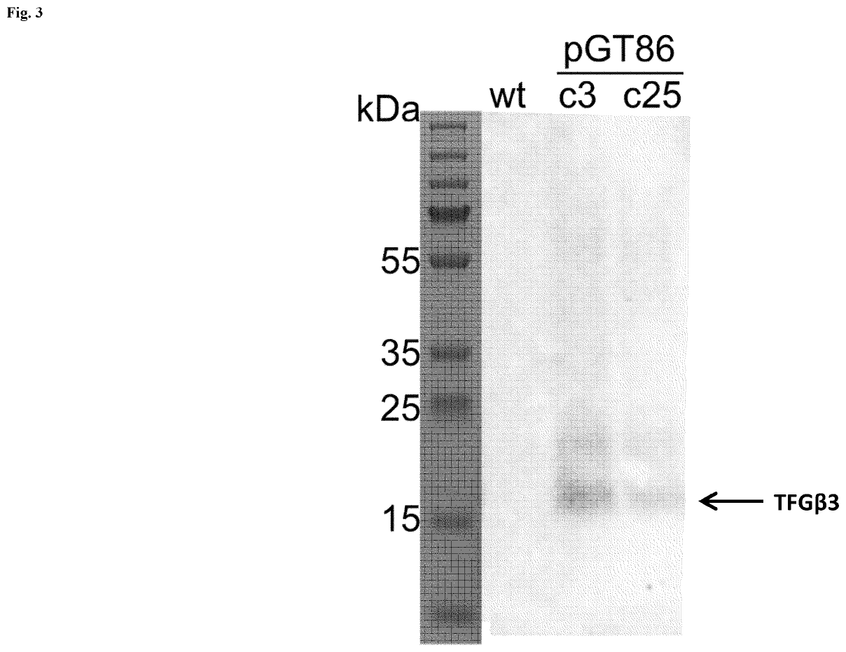

The results are shown in FIGS. 2 and 3.

As seen in FIGS. 2 and 3, for both colonies assayed (c3 and c25), expression of TGFβ3 is seen, showing that successful integration and expression in Pichia pastoris has been achieved.

3.4% of clones express TGFβ3.

Concentration was assessed for TGFβ3 colony c3, because it gave the strongest band on western blot (FIG. 3). TGFβ3 concentration was established using a calibration curve to enable quantitative dotblot. A calibration curve was generated by plotting the dot intensity against known protein concentration. Dot intensity was measured using ImageJ. The intensity of TGFβ3 c3 dot was measured (using ImageJ) and compared using y=mx+c to the calibration curve to establish concentration.

The concentration of TGFβ3 was 8.49 mg/L.

### Example 4: Plasmid Assembly 2

Escherichia coli strain DH5α was used for all DNA assembly and all other cloning described. E. coli DH5α cells were routinely grown in LB medium (tryptone 10 g l⁻¹, yeast extract 5 g l⁻¹ and NaCl 5 g l⁻¹) at 37° C., with shaking at 225 rpm, or on LB agar plates (containing 15 g l⁻¹ bacteriological agar) at 37° C. LB was supplemented with kanamycin (50 μg ml⁻¹) or chloramphenicol (25 μg ml⁻¹) as appropriate. Chemically competent E. coli DH5α cells were routinely used for transformation.

Previously assembled plasmids used in this study are as described in Obst el al. “A Modular Toolkit for Generating Pichia pastoris Secretion Libraries”, ACS Synth. Biol., 2017 and in Lee et al. “A Highly Characterized Yeast Toolkit for Modular, Multipart Assembly”, ACS Synth. Biol., 2015.

Plasmid pBM64 (SEQ ID NO: 16) was assembled from five individual parts: Paox1 (pPTK001), α-MF (pPTK005), TGFβ3 CDS2 (pGT102), TEV_His_Tag_Linker and Taox1 (pMMp010), into the backbone pMMc002. The Golden Gate reaction was performed as described in Taylor et al. “Start-Stop Assembly: a functionally scarless DNA assembly system optimized for metabolic engineering”, Nucleic Acids Res., 2019. Chemically competent E. coli cells were transformed with 10 μl of the pBM64 Golden Gate assembly reaction. The plasmids of transformant colonies were purified and sequence verified.

Plasmid pBM65 (SEQ ID NO: 17) was assembled from five individual parts: Paox1 (pPTK001), α-MF (pPTK005), TGFβ3 CDS3 (pGT103), TEV_His_Tag_Linker and Taox1 (pMMp010), into the backbone pMMc002. The Golden Gate reaction was performed as described in Taylor et al. “Start-Stop Assembly: a functionally scarless DNA assembly system optimized for metabolic engineering”, Nucleic Acids Res., 2019. Chemically competent E. coli cells were transformed with 10 μl of the pPM65 Golden Gate assembly reaction. The plasmids of transformant colonies were purified and sequence verified.

The TGFβ3 CDS2 (pGT102) was stored in the Level 0 storage plasmid pYTK001.

The TGFβ3 CDS3 (pGT103) was stored in the Level 0 storage plasmid pYTK001.

The coding sequences (CDS2 and CDS3) that encode the human Transforming growth factor beta isoform 3 (TGFβ3) were codon optimised for expression in Pichia pastoris (rather than optimised for the host from which it was derived, as would be normal practice). TGFβ3 CDS2 (SEQ ID NO: 12) was codon optimised using GeneART software (Thermo Fisher Scientific). TGFβ3 CDS3 (SEQ ID NO: 13) was codon optimised using the OPTIMIZER one AA—one codon methodology (http://qenomes.urv.es/OPTIMIZER/). DNA synthesis was carried out by TWIST Biosciences. The synthetic DNA was directly mixed with pYTK001 in a Golden Gate reaction using the conditions described by Lee et al. “A Highly Characterized Yeast Toolkit for Modular, Multipart Assembly” ACS Sythn Biol., 2015. E. coli was transformed with the resultant reaction mixtures. The plasmids of transformant colonies were purified and sequence verified.

The tag was generated by annealing complementary pairs of oligonucleotides, as described in Taylor et al. 2019. The TEV_His_Tag_Linker (SEQ ID NO: 18) resulted from annealing oGT44 and oGT45 (sequences shown below). The resultant double stranded DNA tags were used directly in Golden Gate reactions.

oGT44
(SEQ ID NO: 14)
ATCCGAAAACCTTTATTTTCAAGGTCATCATCACCACCATCATTAA
oGT45
(SEQ ID NO: 15)
GCCATTAATGATGGTGGTGATGATGACCTTGAAAATAAAGGTTTTC

Taox1 was synthesized as described above in Example 1 and pMMc002 was assembled as described in Example 1.

The resultant plasmids (pBM64 and pBM65) are shown in FIGS. 4A-4D.

### Example 5: Pichia pastoris Transformation 2

Pichia pastoris strain CBS7435 was used for transforming growth factor beta isoform 3 (TGFβ3) expression. P. pastoris cells were routinely grown in YPD medium (1% yeast extract, 2% peptone, 2% dextrose) at 30° C., with shaking at 225 rpm, or on YPD agar plates (containing 15 g l⁻¹ bacteriological agar) at 30° C. YPD was supplemented with Zeocin (100 μg ml⁻¹) as appropriate.

1000 μg of each plasmid (pBM64 and pBM65) were linearised using Pmel (NEB), as per manufacturers instructions. Linearised plasmid was cleaned-up by gel purification using the QlAquick Gel Extraction kit (Qiagen). P. pastoris cells were transformed with 100 μg of linearised plasmid using the condensed protocol for electroporation as described in Lin-Cereghino, J et al. “Condensed protocol for competent cell preparation and transformation of the methylotrophic yeast Pichia pastoris” Biotechniques, 2005.

### Example 6: TGFβ3 Expression 2

P. pastoris protein expression, using each plasmid, was carried out in buffered dextrose/methanol complex medium (BMDY/BMMY; 1% yeast extract, 2% peptone, 100 mM potassium phosphate (pH 6.0), 1.34% yeast nitrogen base, 4×10⁻⁵% biotin, 2% dextrose or 0.5% methanol).

For high throughput screening of protein expression in P. pastoris deep 96-well plates sealed with sterile breathable sealing film (Breathe Easier, Sigma Aldrich) containing 300 μl of medium were used. For larger scale expression, 500 ml baffled shake flasks containing 100 ml of medium were used. Isolated transformant clones of P. pastoris were used to inoculate YPD supplemented with zeocin and grown at 30° C. at 225 rpm for 24 hours. Cultures were diluted 1:100 into BMDY supplemented with zeocin and grown at 30° C. at 225 rpm for 24 hours. Following incubation, cells were pelleted at 4000 rpm for 5 minutes, supernatant was removed, and fresh BMMY media added to induce protein expression. Cultures were incubated at 30° C., 225 rpm for 48 hours, at 24 hours cultures were supplemented with methanol (100%) to a final concentration 0.5% v/v. Following incubation, cultures were pelleted at 4000 rpm for 5 min, and the supernatants were collected for protein expression assays.

Trichloroacetic acid (100%) was added to supernatant to a final concentration of 10%. Supernatant was incubated at −20° C. for 15 minutes, before protein precipitates were pelleted at 10,000 g, 4° C. for 15 minutes. The pellet was washed twice with acetone (100%) and then air dried. The washed pellet was resuspended in 1/10 of the supernatant volume in Tris-HCl (50 mM, pH 8).

100 μl of supernatant was loaded into individual wells of the Dot Blotter apparatus (Wolflabs). A vacuum was applied to transfer the proteins onto a nitrocellulose membrane (Amersham). TGFβ3 was detected using a 1:1000 dilution of the Mouse anti-HiBiT primary antibody (CS2006A01) followed by 1:2500 dilution of the horseradish peroxidase (HRP) conjugated anti-mouse secondary antibody (W4021). Dots were observed using the ECL Western Blotting Substrate (Promega W1001).

20 μl of sample (supernatant or concentrated supernatant) were denatured by boiling for 10 minutes in reducing SDS sample buffer (4× Laemmli protein sample buffer supplemented with 50 mM DTT). The prestained protein ladder (10-250 kDa, PageRuler, Thermo Scientific) was used to estimate molecular weight. Proteins were resolved by sodium dodecyl sulphate polyacrylamide gel electrophoresis (SDS-PAGE) using 12% Surepage precast gels (GenScript). Proteins were fixed to a nitrocellulose membrane (Amersham) using the wet/tank blotting systems (Bio Rad; 100V, 1 hour, 4° C.). TGFβ3 was detected using a 1:1000 dilution of the Mouse anti-HiBiT primary antibody (Promega CS2006A01) followed by 1:2500 dilution of the horseradish peroxidase (HRP) conjugated anti-mouse secondary antibody (Promega W4021). Dots were observed using the ECL Western Blotting Substrate (Promega W1001).

In this experimental protocol, contrary to usual practice (6-10 verified clones as recommended by the Invitrogen Pichia Expression Kit manual), we screened greater than 50 transformant clones. This was significant because only 6.25% of clones screened expressed TGFβ3.

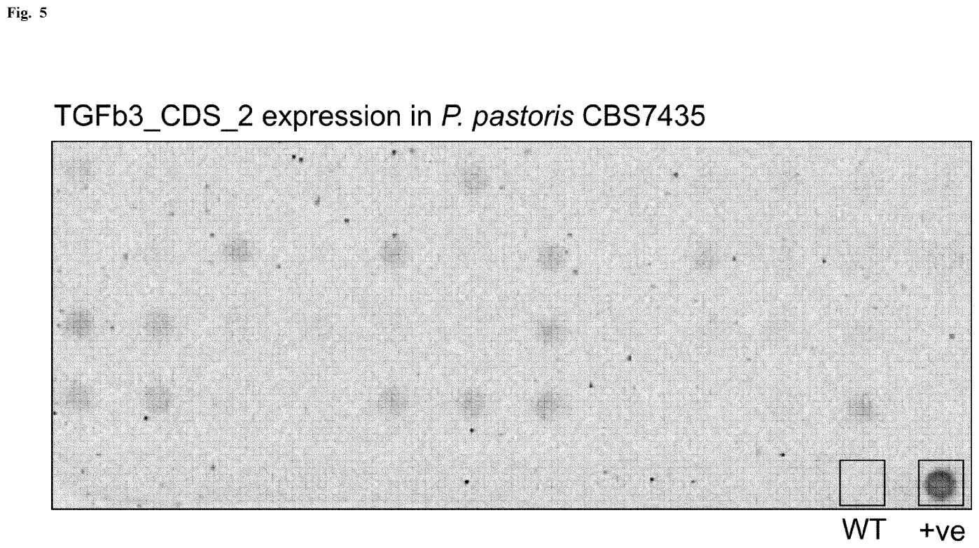

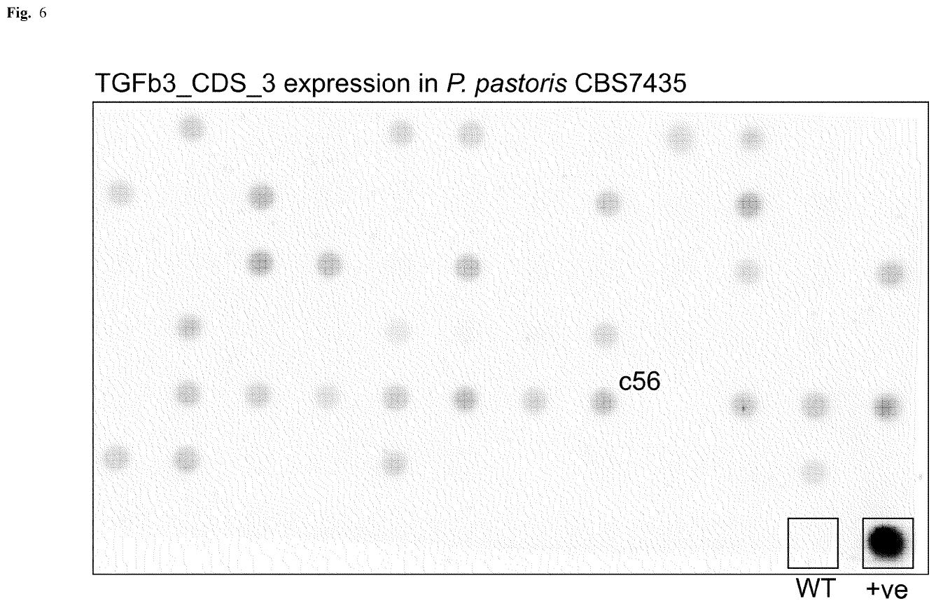

The results are shown in FIGS. 5 and 6.

As seen in FIGS. 5 and 6 expression of TGFβ3 is seen, showing that successful integration and expression in Pichia pastoris has been achieved.

For pBM64 (FIG. 5), 25.9% of clones express TGFβ3. For pBM65 (FIG. 6), 37.8% of clones express TGFβ3.

### Example 7: Liquid PTVA

To further increase TGFβ3 protein concentration, liquid post-transformational vector amplification (PTVA) was used. Liquid PTVA applies increasing selective pressure on Pichia strains, typically via antibiotic concentration, to generate more copies of the inserted foreign DNA that contains the gene of interest as well as an antibiotic marker. The clones that survive at the higher concentrations typically have multiple copies of the antibiotic marker, to accommodate increased antibiotic concentration, and therefore multiple copies of the gene of interest, which leads to increase protein concentration.

Pichia strain BM65c56 (BM102) (FIG. 6) was chosen as the parental clone for Liquid PTVA, which was performed as described in Aw, R and Polizzi, K “Liquid PTVA: a faster and cheaper alternative for generating multi-copy clones in Pichia pastoris”.

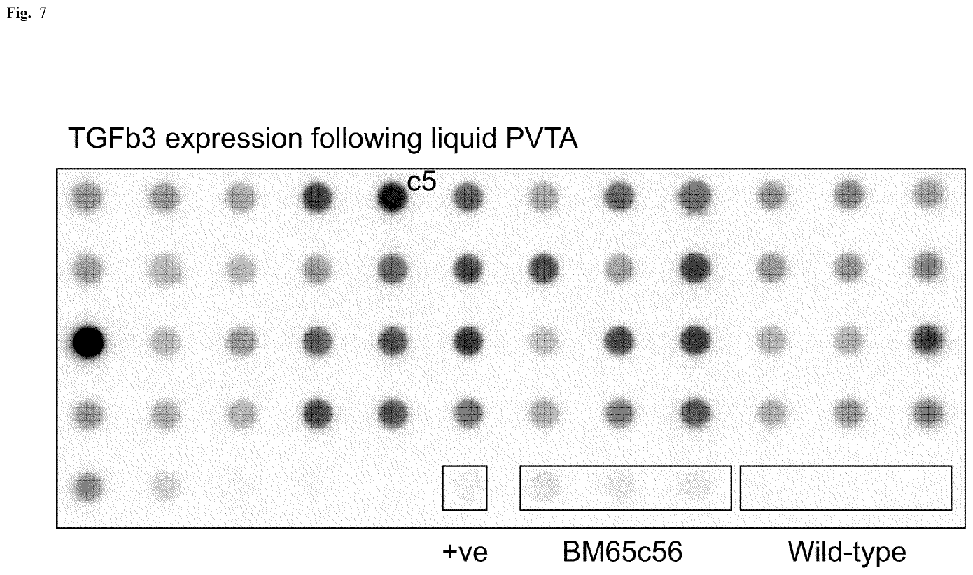

The results are shown in FIG. 7.

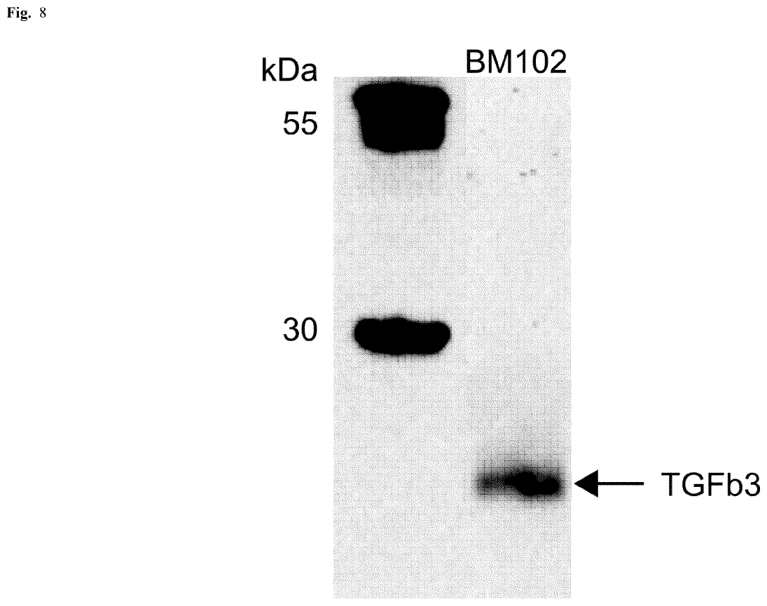

Expression of clone c5 (FIG. 7) was scaled up to 100 ml (as described above in Example 6) as it gave a very strong dot intensity (FIG. 7), the protein was visualised using western blot (FIG. 8) and quantified using the His Tag ELISA Detection Kit (GenScript), in accordance with manufacturer's instructions.

The concentration of TGFβ3 was 17 mg/L.

### Sequences

| SEQ | | | |
|---|---|---|---|
| ID | | | |
| NO: | Description | Sequence | Length |
| 1 | Human | MPPSGLRLLPLLLPLLWLLVLTPGRPAAGLSTCKTIDMEL | 390 |
| | TGFβ1 (aa) | VKRKRIEAIRGQILSKLRLASPPSQGEVPPGPLPEAVLAL | |
| | | YNSTRDRVAGESAEPEPEPEADYYAKEVTRVLMVETHN | |
| | | EIYDKFKQSTHSIYMFFNTSELREAVPEPVLLSRAELRLL | |
| | | RLKLKVEQHVELYQKYSNNSWRYLSNRLLAPSDSPEWL | |
| | | SFDVTGVVRQWLSRGGEIEGFRLSAHCSCDSRDNTLQV | |
| | | DINGFTTGRRGDLATIHGMNRPFLLLMATPLERAQHLQS | |
| | | SRHRRALDTNYCFSSTEKNCCVRQLYIDFRKDLGWKWI | |
| | | HEPKGYHANFCLGPCPYIWSLDTQYSKVLALYNQHNPG | |
| | | ASAAPCCVPQALEPLPIVYYVGRKPKVEQLSNMIVRSCK | |
| | | CS | |
| 2 | Human | MHYCVLSAFLILHLVTVALSLSTCSTLDMDQFMRKRIEAI | 442 |
| | TGFβ2 (aa) | RGQILSKLKLTSPPEDYPEPEEVPPEVISIYNSTRDLLQE | |
| | | KASRRAAACERERSDEEYYAKEVYKIDMPPFFPSETVCP | |
| | | VVTTPSGSVGSLCSRQSQVLCGYLDAIPPTFYRPYFRIV | |
| | | RFDVSAMEKNASNLVKAEFRVFRLQNPKARVPEQRIELY | |
| | | QILKSKDLTSPTQRYIDSKVVKTRAEGEWLSFDVTDAVH | |
| | | EWLHHKDRNLGFKISLHCPCCTFVPSNNYIIPNKSEELEA | |
| | | RFAGIDGTSTYTSGDQKTIKSTRKKNSGKTPHLLLMLLPS | |
| | | YRLESQQTNRRKKRALDAAYCFRNVQDNCCLRPLYIDF | |
| | | KRDLGWKWIHEPKGYNANFCAGACPYLWSSDTQHSRV | |
| | | LSLYNTINPEASASPCCVSQDLEPLTILYYIGKTPKIEQLS | |
| | | NMIVKSCKCS | |
| 3 | Human | ALDTNYCFRNLEENCCVRPLYIDFRQDLGWKWVHEPKG | 112 |
| | TGFβ3 (aa) | YYANFCSGPCPYLRSADTTHSTVLGLYNTLNPEASASPC | |
| | | CVPQDLEPLTILYYVGRTPKVEQLSNMVVKSCKCS | |
| 4 | Human | gcactcgatacgaattactgttttagaaatcttgaagagaactgttgcgtg | 336 |
| | TGFβ3 (nt) | cgtcccctgtacattgatttccgtcaagatctgggatggaaatgggttcatg | |
| | | aaccaaagggttactatgccaacttttgttctggtccttgcccttacctgcgg | |
| | | tcggctgatactactcattccaccgttctggggctgtacaacactctgaatc | |
| | | ctgaagcttccgcctccccatgttgtgtccctcaagacctggagccactg | |
| | | accatcctgtactacgttggacgcacccccaaagtggaacaactctcca | |
| | | acatggttgtcaagagctgcaaatgttca | |
| 5 | AOX1 | gatctaacatccaaagacgaaaggttgaatgaaacctttttgccatccga | 939 |
| | promoter (nt) | catccacaggtccattctcacacataagtgccaaacgcaacaggaggg | |
| | | gatacactagcagcagaccgttgcaaacgcaggacctccactcctcttc | |
| | | tcctcaacacccacttttgccatcgaaaaaccagcccagttattgggcttg | |
| | | attggagctcgctcattccaattccttctattaggctactaacaccatgacttt | |
| | | attagcctgtctatcctggcccccctggcgaggttcatgtttgtttatttccga | |
| | | atgcaacaagctccgcattacacccgaacatcactccagatgagggctt | |
| | | tctgagtgtggggtcaaatagtttcatgttccccaaatggcccaaaactga | |
| | | cagtttaaacgctgtcttggaacctaatatgacaaaagcgtgatctcatcc | |
| | | aagatgaactaagtttggttcgttgaaatgctaacggccagttggtcaaa | |
| | | aagaaacttccaaaagtcggcataccgtttgtcttgtttggtattgattgac | |
| | | gaatgctcaaaaataatctcattaatgcttagcgcagtctctctatcgcttct | |
| | | gaaccccggtgcacctgtgccgaaacgcaaatggggaaacacccgct | |
| | | ttttggatgattatgcattgtctccacattgtatgcttccaagattctggtggg | |
| | | aatactgctgatagcctaacgttcatgatcaaaatttaactgttctaacccc | |
| | | tacttgacagcaatatataaacagaaggaagctgccctgtcttaaaccttt | |
| | | ttttttatcatcattattagcttactttcataattgcgactggttccaattgacaa | |
| | | gcttttgattttaacgacttttaacgacaacttgagaagatcaaaaaacaa | |
| | | ctaattattcgaaacg | |
| 6 | Alpha mating- | atgagatttccttcaatttttactgctgttttattcgcagcatcctccgcattag | 267 |
| | factor (nt) | ctgctccagtcaacactacaacagaagatgaaacggcacaaattccg | |
| | | gctgaagctgtcatcggttactcagatttagaaggggatttcgatgttgctg | |
| | | ttttgccattttccaacagcacaaataacgggttattgtttataaatactacta | |
| | | ttgccagcattgctgctaaagaagaaggggtatctctcgagaaaagag | |
| | | aggctgaagct | |
| 7 | AOX1 | tcaagaggatgtcagaatgccatttgcctgagagatgcaggcttcattttt | 247 |
| | terminator | gatacttttttatttgtaacctatatagtataggattttttttgtcattttgtttcttctc | |
| | (nt) | gtacgagcttgctcctgatcagcctatctcgcagctgatgaatatcttgtgg | |
| | | taggggtttgggaaaatcattcgagtttgatgtttttcttggtatttcccactcc | |
| | | tcttcagagtacagaagattaagtgaga | |
| 8 | His-tag fused | catcatcatcatcatcatgtgagcggctggcggctgttcaagaagattag | 51 |
| | to HiBit tag | c | |
| | (nt) | | |
| 9 | pGT86 | cgtgcggccgcccctgaattcgcatctagatggtagagccacaaacag | 5161 |
| | plasmid | ccggtacaagcaacgatctccaggaccatctgaatcatgcgcggatga | |
| | sequence (nt) | cacgaactcacgacggcgatcacagacattaacccacagtacagaca | |
| | | ctgcgacaacgtggcaattcgtcgcaataccgtctcactgaactggccg | |
| | | ataattgcagacgaacggatctaacatccaaagacgaaaggttgaatg | |
| | | aaacctttttgccatccgacatccacaggtccattctcacacataagtgcc | |
| | | aaacgcaacaggaggggatacactagcagcagaccgttgcaaacgc | |
| | | aggacctccactcctcttctcctcaacacccacttttgccatcgaaaaacc | |
| | | agcccagttattgggcttgattggagctcgctcattccaattccttctattag | |
| | | gctactaacaccatgactttattagcctgtctatcctggcccccctggcga | |
| | | ggttcatgtttgtttatttccgaatgcaacaagctccgcattacacccgaac | |
| | | atcactccagatgagggctttctgagtgtggggtcaaatagtttcatgttcc | |
| | | ccaaatggcccaaaactgacagtttaaacgctgtcttggaacctaatatg | |
| | | acaaaagcgtgatctcatccaagatgaactaagtttggttcgttgaaatg | |
| | | ctaacggccagttggtcaaaaagaaacttccaaaagtcggcataccgtt | |
| | | tgtcttgtttggtattgattgacgaatgctcaaaaataatctcattaatgctta | |
| | | gcgcagtctctctatcgcttctgaaccccggtgcacctgtgccgaaacgc | |
| | | aaatggggaaacacccgctttttggatgattatgcattgtctccacattgtat | |
| | | gcttccaagattctggtgggaatactgctgatagcctaacgttcatgatca | |
| | | aaatttaactgttctaacccctacttgacagcaatatataaacagaagga | |
| | | agctgccctgtcttaaacctttttttttatcatcattattagcttactttcataattg | |
| | | cgactggttccaattgacaagcttttgattttaacgacttttaacgacaactt | |
| | | gagaagatcaaaaaacaactaattattcgaaacgtatgagatttccttca | |
| | | atttttactgctgttttattcgcagcatcctccgcattagctgctccagtcaac | |
| | | actacaacagaagatgaaacggcacaaattccggctgaagctgtcatc | |
| | | ggttactcagatttagaaggggatttcgatgttgctgttttgccattttccaac | |
| | | agcacaaataacgggttattgtttataaatactactattgccagcattgctg | |
| | | ctaaagaagaaggggtatctctcgagaaaagagaggctgaagctggtt | |
| | | ctgcactcgatacgaattactgttttagaaatcttgaagagaactgttgcgt | |
| | | gcgtcccctgtacattgatttccgtcaagatctgggatggaaatgggttcat | |
| | | gaaccaaagggttactatgccaacttttgttctggtccttgcccttacctgcg | |
| | | gtcggctgatactactcattccaccgttctggggctgtacaacactctgaat | |
| | | cctgaagcttccgcctccccatgttgtgtccctcaagacctggagccactg | |
| | | accatcctgtactacgttggacgcacccccaaagtggaacaactctcca | |
| | | acatggttgtcaagagctgcaaatgttcaggatcccatcatcatcatcatc | |
| | | atgtgagcggctggcggctgttcaagaagattagctaatggctcaagag | |
| | | gatgtcagaatgccatttgcctgagagatgcaggcttcatttttgatacttttt | |
| | | tatttgtaacctatatagtataggattttttttgtcattttgtttcttctcgtacgag | |
| | | cttgctcctgatcagcctatctcgcagctgatgaatatcttgtggtaggggtt | |
| | | tgggaaaatcattcgagtttgatgtttttcttggtatttcccactcctcttcaga | |
| | | gtacagaagattaagtgagagctgagcatgagacggaaatctgctcgt | |
| | | cagtggtgctcacactgacgaatcatgtacagatcataccgatgactgc | |
| | | ctggcgactcacaactaagcaagacagccggaaccagcgccggcga | |
| | | acaccactgcatatatggcatatcacaacagtccaactagtgcactgca | |
| | | gtacagtttagcttgcctcgtccccgccgggtcacccggccagcgacat | |
| | | ggaggcccagaataccctccttgacagtcttgacgtgcgcagctcaggg | |
| | | gcatgatgtgactgtcgcccgtacatttagcccatacatccccatgtataat | |
| | | catttgcatccatacattttgatggccgcacggcgcgaagcaaaaattac | |
| | | ggctcctcgctccagacctgcgagcagggaaacgctcccctcacagac | |
| | | gcgttgaattgtccccacgccgcgcccctgtagagaaatataaaaggtt | |
| | | aggatttgccactgaggttcttctttcatatacttccttttaaaatcttgctagg | |
| | | atacagttctcacatcacatccgaacataaacaaaaatggctaaattaa | |
| | | catctgccgttcctgttttaacagctagggatgttgcaggtgctgtagagtttt | |
| | | ggacagataggttaggattctcaagagactttgttgaggacgattttgctg | |
| | | gtgttgtcagggatgacgttactttatttatctcagcagtccaagatcaagtt | |
| | | gtccctgataatacattggcttgggtctgggtcaggggtttagatgaattat | |
| | | atgctgaatggtcagaagttgtatctacaaacttcagagatgcttctggtcc | |
| | | agctatgaccgagattggtgaacagccatggggtagagaatttgctttga | |
| | | gagatccagctggaaattgtgttcattttgttgctgaagaacaagattaaa | |
| | | gtaactgacaataaaaagattcttgttttcaagaacttgtcatttgtatagtttt | |
| | | tttatattgtagttgttctattttaatcaaatgttagcgtgatttatattttttttcgcc | |
| | | tcgacatcatctgcccagatgcgaagttaagtgcgcagaaagtaatatc | |
| | | atgcgtcaatcgtatgtgaatgctggtcgctatactggagttggccgtggc | |
| | | cgtgctcgtcctcgtcggccggcttgtcgacgacggcggtcaccgtcgtc | |
| | | aggatcatccgggccacaagcttgctgacagaagcctcaagaaaaaa | |
| | | aaaattcttcttcgactatgctggaggcagagatgatcgagccggtagtt | |
| | | aactatatatagctaaattggttccatcacccgagcggccgcgtgttaca | |
| | | accaattaaccaattctgattagaaaaactcatcgagcatcaaatgaaa | |
| | | ctgcaatttattcatatcaggattatcaataccatatttttgaaaaagccgttt | |
| | | ctgtaatgaaggagaaaactcaccgaggcagttccataggatggcaa | |
| | | gatcctggtatcggtctgcgattccgactcgtccaacatcaatacaaccta | |
| | | ttaatttcccctcgtcaaaaataaggttatcaagtgagaaatcaccatgag | |
| | | tgacgactgaatccggtgagaatggcaaaagcttatgcatttctttccaga | |
| | | cttgttcaacaggccagccattacgctcgtcatcaaaatcactcgcatca | |
| | | accaaaccgttattcattcgtgattgcgcctgagcgaggcgaaatacgc | |
| | | gatcgctgttaaaaggacaattacaaacaggaatcgaatgcaaccggc | |
| | | gcaggaacactgccagcgcatcaacaatattttcacctgaatcaggata | |
| | | ttcttctaatacctggaatgctgttttcccggggatcgcagtggtgagtaac | |
| | | catgcatcatcaggagtacggataaaatgcttgatggtcggaagaggc | |
| | | ataaattccgtcagccagtttagtctgaccatctcatctgtaacatcattgg | |
| | | caacgctacctttgccatgtttcagaaacaactctggcgcatcgggcttcc | |
| | | catacaatcgatagattgtcgcacctgattgcccgacattatcgcgagcc | |
| | | catttatacccatataaatcagcatccatgttggaatttaatcgcggcctgg | |
| | | agcaagacgtttcccgttgaatatggctcataacaccccttgtattactgttt | |
| | | atgtaagcagacagttttattgttcatgatgatatatttttatcttgtgcaatgta | |
| | | acatcagagattttgagacacaacgtggctttgttgaataaatcgaactttt | |
| | | gctgagttgaaggatcagtcatgaccaaaatcccttaacgtgagttttcgtt | |
| | | ccactgagcgtcagaccccgtagaaaagatcaaaggatcttcttgagat | |
| | | cctttttttctgcgcgtaatctgctgcttgcaaacaaaaaaaccaccgctac | |
| | | cagcggtggtttgtttgccggatcaagagctaccaactctttttccgaaggt | |
| | | aactggcttcagcagagcgcagataccaaatactgttcttctagtgtagc | |
| | | cgtagttaggccaccacttcaagaactctgtagcaccgcctacatacctc | |
| | | gctctgctaatcctgttaccagtggctgctgccagtggcgataagtcgtgt | |
| | | cttaccgggttggactcaagacgatagttaccggataaggcgcagcggt | |
| | | cgggctgaacggggggttcgtgcacacagcccagcttggagcgaacg | |
| | | acctacaccgaactgagatacctacagcgtgagctatgagaaagcgc | |
| | | cacgcttcccgaagggagaaaggcggacaggtatccggtaagcggc | |
| | | agggtcggaacaggagagcgcacgagggagcttccagggggaaac | |
| | | gcctggtatctttatagtcctgtcgggtttcgccacctctgacttgagcgtcg | |
| | | atttttgtgatgctcgtcaggggggcggagcctatggaaaaacgccagc | |
| | | aacgcggcctttttacggttcctggccttttgctggccttttgctcacatgttctt | |
| | | tcctgcgttatcccctgattctgtggataac | |
| 10 | oBM37 | gcatcgtctcatcggtctcaatcccatcatcatcatcatcatgtgagcggct | 97 |
| | oligonucleotide | ggcggctgttcaagaagattagctggctgagacctgagacggcat | |
| | (nt) | | |
| 11 | oBM38 | atgccgtctcaggtctcagccattagctaatcttcttgaacagccgccagc | 100 |
| | oligonucleotide | cgctcacatgatgatgatgatgatgggattgagaccgatgagacgatgc | |
| | (nt) | | |
| 12 | Human | gctttggacaccaactactgcttcagaaacctggaagagaactgttgcgt | 336 |
| | TGFβ3 (nt)- | cagaccactgtacatcgacttcagacaagaccttggttggaagtgggttc | |
| | CDS2 | acgaacctaagggttactacgccaacttctgttctggtccatgtccatactt | |
| | | gagatccgctgacactactcactctaccgttttgggtctgtacaacactttg | |
| | | aacccagaggcttctgcttccccatgttgtgttccacaagacttggagcca | |
| | | ttgaccatcttgtactacgttggtagaaccccaaaggtcgagcagttgtcc | |
| | | aacatggttgttaagtcctgcaagtgttct | |
| 13 | Human | gctttggatactaactactgttttagaaacttggaagaaaactgttgtgttag | 336 |
| | TGFβ3 (nt)- | accattgtacattgattttagacaagatttgggttggaagtgggttcatgaa | |
| | CDS3 | ccaaagggttactacgctaacttttgttctggtccatgtccatacttgagatc | |
| | | tgctgatactactcattctactgttttgggtttgtacaacactttgaacccaga | |
| | | agcttctgcttctccatgttgtgttccacaagatttggaaccattgactattttg | |
| | | tactacgttggtagaactccaaaggttgaacaattgtctaacatggttgtta | |
| | | agtcttgtaagtgttct | |
| 14 | oGT44 | atccgaaaacctttattttcaaggtcatcatcaccaccatcattaa | 46 |
| | oligonucleotide | | |
| | (nt) | | |
| 15 | oGT45 | gccattaatgatggtggtgatgatgaccttgaaaataaaggttttc | 46 |
| | oligonucleotide | | |
| | (nt) | | |
| 16 | pBM64 | cgtgcggccgcccctgaattcgcatctagatggtagagccacaaacag | 5149 |
| | plasmid | ccggtacaagcaacgatctccaggaccatctgaatcatgcgcggatga | |
| | sequence (nt) | cacgaactcacgacggcgatcacagacattaacccacagtacagaca | |
| | | ctgcgacaacgtggcaattcgtcgcaataccgtctcactgaactggccg | |
| | | ataattgcagacgaacggatctaacatccaaagacgaaaggttgaatg | |
| | | aaacctttttgccatccgacatccacaggtccattctcacacataagtgcc | |
| | | aaacgcaacaggaggggatacactagcagcagaccgttgcaaacgc | |
| | | aggacctccactcctcttctcctcaacacccacttttgccatcgaaaaacc | |
| | | agcccagttattgggcttgattggagctcgctcattccaattccttctattag | |
| | | gctactaacaccatgactttattagcctgtctatcctggcccccctggcga | |
| | | ggttcatgtttgtttatttccgaatgcaacaagctccgcattacacccgaac | |
| | | atcactccagatgagggctttctgagtgtggggtcaaatagtttcatgttcc | |
| | | ccaaatggcccaaaactgacagtttaaacgctgtcttggaacctaatatg | |
| | | acaaaagcgtgatctcatccaagatgaactaagtttggttcgttgaaatg | |
| | | ctaacggccagttggtcaaaaagaaacttccaaaagtcggcataccgtt | |
| | | tgtcttgtttggtattgattgacgaatgctcaaaaataatctcattaatgctta | |
| | | gcgcagtctctctatcgcttctgaaccccggtgcacctgtgccgaaacgc | |
| | | aaatggggaaacacccgctttttggatgattatgcattgtctccacattgtat | |
| | | gcttccaagattctggtgggaatactgctgatagcctaacgttcatgatca | |
| | | aaatttaactgttctaacccctacttgacagcaatatataaacagaagga | |
| | | agctgccctgtcttaaacctttttttttatcatcattattagcttactttcataattg | |
| | | cgactggttccaattgacaagcttttgattttaacgacttttaacgacaactt | |
| | | gagaagatcaaaaaacaactaattattcgaaacgtatgagatttccttca | |
| | | atttttactgctgttttattcgcagcatcctccgcattagctgctccagtcaac | |
| | | actacaacagaagatgaaacggcacaaattccggctgaagctgtcatc | |
| | | ggttactcagatttagaaggggatttcgatgttgctgttttgccattttccaac | |
| | | agcacaaataacgggttattgtttataaatactactattgccagcattgctg | |
| | | ctaaagaagaaggggtatctctcgagaaaagagaggctgaagctggtt | |
| | | ctgctttggacaccaactactgcttcagaaacctggaagagaactgttgc | |
| | | gtcagaccactgtacatcgacttcagacaagaccttggttggaagtgggt | |
| | | tcacgaacctaagggttactacgccaacttctgttctggtccatgtccatac | |
| | | ttgagatccgctgacactactcactctaccgttttgggtctgtacaacacttt | |
| | | gaacccagaggcttctgcttccccatgttgtgttccacaagacttggagcc | |
| | | attgaccatcttgtactacgttggtagaaccccaaaggtcgagcagttgtc | |
| | | caacatggttgttaagtcctgcaagtgttctggatccgaaaacctttattttc | |
| | | aaggtcatcatcaccaccatcattaatggctcaagaggatgtcagaatg | |
| | | ccatttgcctgagagatgcaggcttcatttttgatacttttttatttgtaacctat | |
| | | atagtataggattttttttgtcattttgtttcttctcgtacgagcttgctcctgatca | |
| | | gcctatctcgcagctgatgaatatcttgtggtaggggtttgggaaaatcatt | |
| | | cgagtttgatgtttttcttggtatttcccactcctcttcagagtacagaagatta | |
| | | agtgagagctgagcatgagacggaaatctgctcgtcagtggtgctcaca | |
| | | ctgacgaatcatgtacagatcataccgatgactgcctggcgactcacaa | |
| | | ctaagcaagacagccggaaccagcgccggcgaacaccactgcatat | |
| | | atggcatatcacaacagtccaactagtgcactgcagtacagtttagcttg | |
| | | cctcgtccccgccgggtcacccggccagcgacatggaggcccagaat | |
| | | accctccttgacagtcttgacgtgcgcagctcaggggcatgatgtgactg | |
| | | tcgcccgtacatttagcccatacatccccatgtataatcatttgcatccata | |
| | | cattttgatggccgcacggcgcgaagcaaaaattacggctcctcgctcc | |
| | | agacctgcgagcagggaaacgctcccctcacagacgcgttgaattgtc | |
| | | cccacgccgcgcccctgtagagaaatataaaaggttaggatttgccact | |
| | | gaggttcttctttcatatacttccttttaaaatcttgctaggatacagttctcac | |
| | | atcacatccgaacataaacaaaaatggctaaattaacatctgccgttcct | |
| | | gttttaacagctagggatgttgcaggtgctgtagagttttggacagataggt | |
| | | taggattctcaagagactttgttgaggacgattttgctggtgttgtcagggat | |
| | | gacgttactttatttatctcagcagtccaagatcaagttgtccctgataatac | |
| | | attggcttgggtctgggtcaggggtttagatgaattatatgctgaatggtca | |
| | | gaagttgtatctacaaacttcagagatgcttctggtccagctatgaccgag | |
| | | attggtgaacagccatggggtagagaatttgctttgagagatccagctgg | |
| | | aaattgtgttcattttgttgctgaagaacaagattaaagtaactgacaataa | |
| | | aaagattcttgttttcaagaacttgtcatttgtatagtttttttatattgtagttgttc | |
| | | tattttaatcaaatgttagcgtgatttatattttttttcgcctcgacatcatctgcc | |
| | | cagatgcgaagttaagtgcgcagaaagtaatatcatgcgtcaatcgtat | |
| | | gtgaatgctggtcgctatactggagttggccgtggccgtgctcgtcctcgt | |
| | | cggccggcttgtcgacgacggcggtcaccgtcgtcaggatcatccggg | |
| | | ccacaagcttgctgacagaagcctcaagaaaaaaaaaattcttcttcga | |
| | | ctatgctggaggcagagatgatcgagccggtagttaactatatatagcta | |
| | | aattggttccatcacccgagcggccgcgtgttacaaccaattaaccaatt | |
| | | ctgattagaaaaactcatcgagcatcaaatgaaactgcaatttattcatat | |
| | | caggattatcaataccatatttttgaaaaagccgtttctgtaatgaaggag | |
| | | aaaactcaccgaggcagttccataggatggcaagatcctggtatcggtc | |
| | | tgcgattccgactcgtccaacatcaatacaacctattaatttcccctcgtca | |
| | | aaaataaggttatcaagtgagaaatcaccatgagtgacgactgaatcc | |
| | | ggtgagaatggcaaaagcttatgcatttctttccagacttgttcaacaggc | |
| | | cagccattacgctcgtcatcaaaatcactcgcatcaaccaaaccgttatt | |
| | | cattcgtgattgcgcctgagcgaggcgaaatacgcgatcgctgttaaaa | |
| | | ggacaattacaaacaggaatcgaatgcaaccggcgcaggaacactg | |
| | | ccagcgcatcaacaatattttcacctgaatcaggatattcttctaatacctg | |
| | | gaatgctgttttcccggggatcgcagtggtgagtaaccatgcatcatcag | |
| | | gagtacggataaaatgcttgatggtcggaagaggcataaattccgtcag | |
| | | ccagtttagtctgaccatctcatctgtaacatcattggcaacgctacctttgc | |
| | | catgtttcagaaacaactctggcgcatcgggcttcccatacaatcgatag | |
| | | attgtcgcacctgattgcccgacattatcgcgagcccatttatacccatata | |
| | | aatcagcatccatgttggaatttaatcgcggcctggagcaagacgtttcc | |
| | | cgttgaatatggctcataacaccccttgtattactgtttatgtaagcagaca | |
| | | gttttattgttcatgatgatatatttttatcttgtgcaatgtaacatcagagatttt | |
| | | gagacacaacgtggctttgttgaataaatcgaacttttgctgagttgaagg | |
| | | atcagtcatgaccaaaatcccttaacgtgagttttcgttccactgagcgtc | |
| | | agaccccgtagaaaagatcaaaggatcttcttgagatcctttttttctgcgc | |
| | | gtaatctgctgcttgcaaacaaaaaaaccaccgctaccagcggtggttt | |
| | | gtttgccggatcaagagctaccaactctttttccgaaggtaactggcttca | |
| | | gcagagcgcagataccaaatactgttcttctagtgtagccgtagttaggc | |
| | | caccacttcaagaactctgtagcaccgcctacatacctcgctctgctaatc | |
| | | ctgttaccagtggctgctgccagtggcgataagtcgtgtcttaccgggttg | |
| | | gactcaagacgatagttaccggataaggcgcagcggtcgggctgaac | |
| | | ggggggttcgtgcacacagcccagcttggagcgaacgacctacaccg | |
| | | aactgagatacctacagcgtgagctatgagaaagcgccacgcttcccg | |
| | | aagggagaaaggcggacaggtatccggtaagcggcagggtcggaa | |
| | | caggagagcgcacgagggagcttccagggggaaacgcctggtatcttt | |
| | | atagtcctgtcgggtttcgccacctctgacttgagcgtcgatttttgtgatgct | |
| | | cgtcaggggggggagcctatggaaaaacgccagcaacgcggccttt | |
| | | ttacggttcctggccttttgctggccttttgctcacatgttctttcctgcgttatcc | |
| | | cctgattctgtggataac | |
| 17 | pBM65 | cgtgcggccgcccctgaattcgcatctagatggtagagccacaaacag | 5149 |
| | plasmid | ccggtacaagcaacgatctccaggaccatctgaatcatgcgcggatga | |
| | sequence (nt) | cacgaactcacgacggcgatcacagacattaacccacagtacagaca | |
| | | ctgcgacaacgtggcaattcgtcgcaataccgtctcactgaactggccg | |
| | | ataattgcagacgaacggatctaacatccaaagacgaaaggttgaatg | |
| | | aaacctttttgccatccgacatccacaggtccattctcacacataagtgcc | |
| | | aaacgcaacaggaggggatacactagcagcagaccgttgcaaacgc | |
| | | aggacctccactcctcttctcctcaacacccacttttgccatcgaaaaacc | |
| | | agcccagttattgggcttgattggagctcgctcattccaattccttctattag | |
| | | gctactaacaccatgactttattagcctgtctatcctggcccccctggcga | |
| | | ggttcatgtttgtttatttccgaatgcaacaagctccgcattacacccgaac | |
| | | atcactccagatgagggctttctgagtgtggggtcaaatagtttcatgttcc | |
| | | ccaaatggcccaaaactgacagtttaaacgctgtcttggaacctaatatg | |
| | | acaaaagcgtgatctcatccaagatgaactaagtttggttcgttgaaatg | |
| | | ctaacggccagttggtcaaaaagaaacttccaaaagtcggcataccgtt | |
| | | tgtcttgtttggtattgattgacgaatgctcaaaaataatctcattaatgctta | |
| | | gcgcagtctctctatcgcttctgaaccccggtgcacctgtgccgaaacgc | |
| | | aaatggggaaacacccgctttttggatgattatgcattgtctccacattgtat | |
| | | gcttccaagattctggtgggaatactgctgatagcctaacgttcatgatca | |
| | | aaatttaactgttctaacccctacttgacagcaatatataaacagaagga | |
| | | agctgccctgtcttaaacctttttttttatcatcattattagcttactttcataattg | |
| | | cgactggttccaattgacaagcttttgattttaacgacttttaacgacaactt | |
| | | gagaagatcaaaaaacaactaattattcgaaacgtatgagatttccttca | |
| | | atttttactgctgttttattcgcagcatcctccgcattagctgctccagtcaac | |
| | | actacaacagaagatgaaacggcacaaattccggctgaagctgtcatc | |
| | | ggttactcagatttagaaggggatttcgatgttgctgttttgccattttccaac | |
| | | agcacaaataacgggttattgtttataaatactactattgccagcattgctg | |
| | | ctaaagaagaaggggtatctctcgagaaaagagaggctgaagctggtt | |
| | | ctgctttggatactaactactgttttagaaacttggaagaaaactgttgtgtt | |
| | | agaccattgtacattgattttagacaagatttgggttggaagtgggttcatg | |
| | | aaccaaagggttactacgctaacttttgttctggtccatgtccatacttgag | |
| | | atctgctgatactactcattctactgttttgggtttgtacaacactttgaaccc | |
| | | agaagcttctgcttctccatgttgtgttccacaagatttggaaccattgacta | |
| | | ttttgtactacgttggtagaactccaaaggttgaacaattgtctaacatggtt | |
| | | gttaagtcttgtaagtgttctggatccgaaaacctttattttcaaggtcatcat | |
| | | caccaccatcattaatggctcaagaggatgtcagaatgccatttgcctga | |
| | | gagatgcaggcttcatttttgatacttttttatttgtaacctatatagtataggat | |
| | | tttttttgtcattttgtttcttctcgtacgagcttgctcctgatcagcctatctcgca | |
| | | gctgatgaatatcttgtggtaggggtttgggaaaatcattcgagtttgatgtt | |
| | | tttcttggtatttcccactcctcttcagagtacagaagattaagtgagagctg | |
| | | agcatgagacggaaatctgctcgtcagtggtgctcacactgacgaatca | |
| | | tgtacagatcataccgatgactgcctggcgactcacaactaagcaaga | |
| | | cagccggaaccagcgccggcgaacaccactgcatatatggcatatca | |
| | | caacagtccaactagtgcactgcagtacagtttagcttgcctcgtccccg | |
| | | ccgggtcacccggccagcgacatggaggcccagaataccctccttga | |
| | | cagtcttgacgtgcgcagctcaggggcatgatgtgactgtcgcccgtac | |
| | | atttagcccatacatccccatgtataatcatttgcatccatacattttgatgg | |
| | | ccgcacggcgcgaagcaaaaattacggctcctcgctccagacctgcg | |
| | | agcagggaaacgctcccctcacagacgcgttgaattgtccccacgccg | |
| | | cgcccctgtagagaaatataaaaggttaggatttgccactgaggttcttctt | |
| | | tcatatacttccttttaaaatcttgctaggatacagttctcacatcacatccg | |
| | | aacataaacaaaaatggctaaattaacatctgccgttcctgttttaacagc | |
| | | tagggatgttgcaggtgctgtagagttttggacagataggttaggattctca | |
| | | agagactttgttgaggacgattttgctggtgttgtcagggatgacgttacttt | |
| | | atttatctcagcagtccaagatcaagttgtccctgataatacattggcttgg | |
| | | gtctgggtcaggggtttagatgaattatatgctgaatggtcagaagttgtat | |
| | | ctacaaacttcagagatgcttctggtccagctatgaccgagattggtgaa | |
| | | cagccatggggtagagaatttgctttgagagatccagctggaaattgtgtt | |
| | | cattttgttgctgaagaacaagattaaagtaactgacaataaaaagattct | |
| | | tgttttcaagaacttgtcatttgtatagtttttttatattgtagttgttctattttaatc | |
| | | aaatgttagcgtgatttatattttttttcgcctcgacatcatctgcccagatgc | |
| | | gaagttaagtgcgcagaaagtaatatcatgcgtcaatcgtatgtgaatgc | |
| | | tggtcgctatactggagttggccgtggccgtgctcgtcctcgtcggccggc | |
| | | ttgtcgacgacggcggtcaccgtcgtcaggatcatccgggccacaagct | |
| | | tgctgacagaagcctcaagaaaaaaaaaattcttcttcgactatgctgga | |
| | | ggcagagatgatcgagccggtagttaactatatatagctaaattggttcc | |
| | | atcacccgagcggccgcgtgttacaaccaattaaccaattctgattaga | |
| | | aaaactcatcgagcatcaaatgaaactgcaatttattcatatcaggattat | |
| | | caataccatatttttgaaaaagccgtttctgtaatgaaggagaaaactca | |
| | | ccgaggcagttccataggatggcaagatcctggtatcggtctgcgattcc | |
| | | gactcgtccaacatcaatacaacctattaatttcccctcgtcaaaaataag | |
| | | gttatcaagtgagaaatcaccatgagtgacgactgaatccggtgagaat | |
| | | ggcaaaagcttatgcatttctttccagacttgttcaacaggccagccatta | |
| | | cgctcgtcatcaaaatcactcgcatcaaccaaaccgttattcattcgtgatt | |
| | | gcgcctgagcgaggcgaaatacgcgatcgctgttaaaaggacaattac | |
| | | aaacaggaatcgaatgcaaccggcgcaggaacactgccagcgcatc | |
| | | aacaatattttcacctgaatcaggatattcttctaatacctggaatgctgtttt | |
| | | cccggggatcgcagtggtgagtaaccatgcatcatcaggagtacggat | |
| | | aaaatgcttgatggtcggaagaggcataaattccgtcagccagtttagtc | |
| | | tgaccatctcatctgtaacatcattggcaacgctacctttgccatgtttcaga | |
| | | aacaactctggcgcatcgggcttcccatacaatcgatagattgtcgcacc | |
| | | tgattgcccgacattatcgcgagcccatttatacccatataaatcagcatc | |
| | | catgttggaatttaatcgcggcctggagcaagacgtttcccgttgaatatg | |
| | | gctcataacaccccttgtattactgtttatgtaagcagacagttttattgttcat | |
| | | gatgatatatttttatcttgtgcaatgtaacatcagagattttgagacacaac | |
| | | gtggctttgttgaataaatcgaacttttgctgagttgaaggatcagtcatga | |
| | | ccaaaatcccttaacgtgagttttcgttccactgagcgtcagaccccgtag | |
| | | aaaagatcaaaggatcttcttgagatcctttttttctgcgcgtaatctgctgct | |
| | | tgcaaacaaaaaaaccaccgctaccagcggtggtttgtttgccggatca | |
| | | agagctaccaactctttttccgaaggtaactggcttcagcagagcgcag | |
| | | ataccaaatactgttcttctagtgtagccgtagttaggccaccacttcaag | |
| | | aactctgtagcaccgcctacatacctcgctctgctaatcctgttaccagtg | |
| | | gctgctgccagtggcgataagtcgtgtcttaccgggttggactcaagacg | |
| | | atagttaccggataaggcgcagcggtcgggctgaacggggggttcgtg | |
| | | cacacagcccagcttggagcgaacgacctacaccgaactgagatacc | |
| | | tacagcgtgagctatgagaaagcgccacgcttcccgaagggagaaag | |
| | | gcggacaggtatccggtaagcggcagggtcggaacaggagagcgca | |
| | | cgagggagcttccagggggaaacgcctggtatctttatagtcctgtcggg | |
| | | tttcgccacctctgacttgagcgtcgatttttgtgatgctcgtcaggggggc | |
| | | ggagcctatggaaaaacgccagcaacgcggcctttttacggttcctggc | |
| | | cttttgctggccttttgctcacatgttctttcctgcgttatcccctgattctgtgga | |
| | | taac | |
| 18 | TEV_His_Tag_ | gaaaacctttattttcaaggtcatcatcaccaccatcattaa | 42 |
| | Linker (nt) | | |

1. A methylotrophic yeast cell whose genome comprises a sequence encoding transforming growth factor beta (TGFβ).

2. The yeast cell of claim 1, wherein the yeast cell is a Pichia pastoris cell.

3. The yeast cell of claim 1 or claim 2, wherein the TGFβ is TGFβ1, TGFβ2 or TGFβ3.

4. The yeast cell of any preceding claim, wherein the TGFβ is TGFβ3, optionally human TGFβ3.

5. The yeast cell of any preceding claim, wherein the sequence comprises the nucleic acid sequence of SEQ ID NO: 13, SEQ ID NO: 12 or SEQ ID NO: 4.

6. The yeast cell of any preceding claim, wherein the sequence comprises the nucleic acid sequence of SEQ ID NO: 13.

7. The yeast cell of any preceding claim, wherein the sequence comprises the nucleic acid sequence of SEQ ID NO: 12.

8. The yeast cell of any preceding claim, wherein the sequence comprises the nucleic acid sequence of SEQ ID NO: 4.

9. The yeast cell of any preceding claim, wherein the yeast cell has been transformed using a plasmid.

10. The yeast cell of claim 9, wherein the plasmid comprises a promoter, the coding sequence, and a terminator.

11. The yeast cell of claim 10, wherein the plasmid additionally comprises a signal peptide.

12. The yeast cell of claim 10 or claim 11, wherein the promoter is an alcohol oxidase I (AOX1) promoter.

13. The yeast cell of any one of claim 10-12, wherein the promoter comprises the nucleic acid sequence of SEQ ID NO: 5.

14. The yeast cell of any one of claims 11-13, wherein the signal peptide is an alpha mating-factor signal peptide.

15. The yeast cell of any one of claims 11-14, wherein the signal peptide comprises the nucleic acid sequence of SEQ ID NO: 6.

16. The yeast cell of any one of claims 10-15, wherein the terminator is an alcohol oxidase I (AOX1) terminator.

17. The yeast cell of any one of claims 10-16, wherein the terminator comprises the nucleic acid sequence of SEQ ID NO: 7.

18. The yeast cell of any one of claims 10-17, wherein the plasmid comprises the nucleic acid sequences of SEQ ID NOs: 4-7.

19. The yeast cell of any one of claims 10-18, wherein the plasmid comprises the nucleic acid sequence of SEQ ID NO: 9.

20. The yeast cell of any one of claims 10-17, wherein the plasmid comprises the nucleic acid sequences of SEQ ID NOs: 12 and 5-7.

21. The yeast cell of any one of claims 10-17 and 20, wherein the plasmid comprises the nucleic acid sequence of SEQ ID NO: 16.

22. The yeast cell of any one of claims 10-17, wherein the plasmid comprises the nucleic acid sequences of SEQ ID NOs: 13 and 5-7.

23. The yeast cell of any one of claims 10-17 and 22, wherein the plasmid comprises the nucleic acid sequence of SEQ ID NO: 17.

24. A methylotrophic yeast cell capable of expressing TGFβ.

25. The yeast cell of claim 24, wherein the yeast cell is capable of expressing TGFβ1, TGFβ2 or TGFβ3.

26. The yeast cell of claim 24 or claim 25, wherein the yeast cell is a Pichia pastoris cell.

27. A polypeptide comprising the amino acid sequence of SEQ ID NO: 3, obtained or obtainable from the yeast cell of any preceding claim.

28. A method of producing a TGFβ polypeptide, wherein the method comprises transforming a methylotrophic yeast cell with a plasmid encoding TGFβ.

29. A method of producing a TGFβ polypeptide, wherein the method comprises cultivating the yeast cell of any of claims 1-26 under conditions which allow for expression of said polypeptide and, optionally, recovering the expressed polypeptide.

30. The method of claim 29, further comprising i) optimising a nucleic acid encoding a TGFβ polypeptide for expression in yeast, optionally methylotrophic yeast, further optionally Pichia pastoris, ii) screening more than ten clones for expression of TGFβ, optionally at least twenty clones, and/or iii) detecting expression with a HiBiT peptide tag or a his-tag.

31. The method of any one of claims 28 to 30, wherein the TGFβ polypeptide is a TGFβ3 polypeptide.

32. A method of growing an animal cell, wherein the method comprises cultivating the animal cell in a growth media containing the polypeptide of claim 27.

33. Use of the polypeptide of claim 27 in an animal cell growth media.
